(19) United States
(12) Patent Application Publication
(10) Pub. No.: US 2012/0295985 A1
(43) Pub. Date: Nov. 22, 2012

(54) METHODS FOR IMPROVING BLOOD GLUCOSE CONTROL

(76) Inventors: Guy M. Miller, Monte Sereno, CA (US); William D. Shrader, Belmont, CA (US); Martin J. Thoolen, San Mateo, CA (US)

(21) Appl. No.: 13/300,449

(22) Filed: Nov. 18, 2011

Related U.S. Application Data
(60) Provisional application No. 61/458,231, filed on Nov. 19, 2010.

(51) Int. Cl. A61K 31/122; A61P 3/08; A61P 3/00; A61P 29/00; A61P 9/00; A61P 9/10; A61P 13/12; C07C 50/28; A61P 3/10

(52) U.S. Cl. 514/690; 552/310; 552/309

(57) ABSTRACT

The present invention is concerned with a method for improving blood glucose control or for preventing or treating a condition requiring increasing insulin sensitivity or reducing insulin resistance in a subject in need of such control which comprises administering to the subject a composition comprising a therapeutically effective amount of one or more quinones of Formula I.

### CROSS-REFERENCE TO RELATED APPLICATIONS

This application claims priority benefit of U.S. Provisional Patent Application No. 61/458,231, filed Nov. 19, 2010. The entire content of that application is hereby incorporated herein by reference.

### FIELD OF THE INVENTION

The present invention is concerned with a method for improving blood glucose control in a subject in need of such control which comprises administering to the subject a composition comprising a therapeutically effective amount of one or more quinones of Formula I. The methods of this invention are particularly applicable to preventing, reducing, ameliorating or treating diabetes, or to stabilize the level of blood sugar, stop the increase in level of blood sugar, reverse the increase in the level of blood sugar, or reduce the level of blood sugar in a subject in need thereof. The methods of this invention are also contemplated for the use in preventing or treating a condition requiring increasing insulin sensitivity or reducing insulin resistance with an effective amount of one or more quinones of Formula I.

### BACKGROUND INFORMATION

Diabetes is a group of metabolic diseases in which a person has high blood sugar caused by the occurrence of abnormal metabolisms of glucose, protein, and lipids. These abnormal metabolisms arise from a deficiency or an insufficiency of the actions of insulin due to either the faulty secretion of insulin by the pancreas or to the cells' inability to respond to the insulin that is produced and to properly use it. Typical signs of diabetes include an abnormal increase in the serum glucose level (i.e., exceeding the normal range of the serum glucose level), and an excretion of glucose in the urine.

Several clinical types of diabetes are recognized, such as Type 1 (diabetes mellitus, insulin-dependent or IDDM); Type 2 (non-insulin-dependent diabetes mellitus); maturity-onset diabetes of the young (MODY); gestational diabetes; and diabetes secondary to other conditions, such as congenital diabetes due to genetic defects of insulin secretion, cystic fibrosis-related diabetes, steroid diabetes induced by high doses of glucocorticoids, and several forms of monogenic diabetes. These subclasses differ in etiology, pathology, genetics, age of onset, and treatment.

Type 1 diabetes, the more severe form of diabetes, accounts for 5 to 10 percent of diabetes and occurs most often in children and young adults. In this form of diabetes the body does not produce any insulin. Without regular injections of insulin the sufferer lapses into a coma and dies. Individuals suffering from Type 1 diabetes are totally insulin dependent.

Type 2 diabetes, the more prevalent type of diabetes, is usually characterized by gradual onset and occurs mainly in people over 40. Type 2 diabetes is a metabolic disorder resulting from the body's inability to make enough insulin or to properly use insulin to meet the body's needs, particularly when the subject is overweight. It is the most common form of the disease. Type 2 diabetes accounts for 90 to 95 percent of diabetes. Type 2 diabetes is nearing epidemic proportions due to a greater prevalence of obesity and sedentary lifestyles. Initially, the combination of dietary measures, weight reduction and oral medication can keep the condition under control for a period of time, but most people with Type 2 diabetes ultimately require insulin injections.

The National Diabetes Information Clearing House suggests that in the United States, 23.6 million people—7.8 percent of the population—have diabetes (see URL diabetes.niddk.nih.gov). Diabetes may be controlled with insulin and in some cases through careful diet, but there is a need for a safe and effective treatment for diabetes with minimal side effects and without the invasive procedure of insulin injection. The complications that arise due to diabetes adversely affect the quality of life of those who suffer from it and result in significant health costs. Therefore there remains a need for improved methods for increasing insulin sensitivity in patients with this and related diseases.

### SUMMARY OF THE INVENTION

In some embodiments, the present invention relates to a composition comprising an effective amount of one or more compounds of Formula I or mixtures thereof and, optionally, a pharmaceutically acceptable carrier, diluent or excipient, for improving blood glucose control in a subject with abnormal glucose tolerance and metabolism in need of such treatment, where the one or more compounds of Formula I have the following structure:

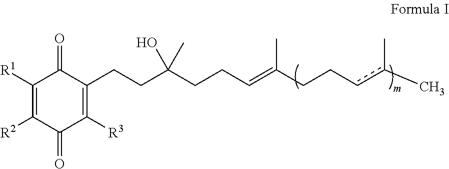

wherein,
each bond indicated by a dashed line can independently be double or single;
R¹, R², R³ are independently of each other hydrogen, (C₁-C₆)alkyl or (C₁-C₆)alkoxy; and
m is an integer of 0 to 12 inclusive;
or any stereoisomer, mixture of stereoisomers, prodrug, metabolite, salt, crystalline form, non-crystalline form, hydrate or solvate thereof. In one embodiment, all of the bonds indicated by a dashed line are single bonds. In another embodiment, all of the bonds indicated by a dashed line are double bonds.

In some embodiments, the composition for improving blood glucose control is administered via oral, gastrointestinal, rectal, parenteral, local, inhalant or enteral route. In other embodiments, the composition for improving blood glucose control is an oral composition.

In some embodiments, the invention relates to a composition for improving blood glucose control comprising an effective amount of one or more compounds of Formula I, wherein R¹ and R² are independently of each other (C₁-C₆)alkoxy and R³ is (C₁-C₆)alkyl. In some embodiments, the invention relates to a composition for improving blood glucose control comprising an effective amount of one or more compounds of Formula I, wherein R¹ and R² are independently of each other (C₁-C₆)alkoxy and R³ is hydrogen. In other embodiments, the invention relates to a composition comprising an effective amount of one or more compounds of Formula I, wherein R¹, R² and R³ are independently of each other (C₁-C₆)alkyl or hydrogen. In other embodiments, the invention relates to a composition comprising an effective amount of one or more compounds of Formula I, wherein R¹, R² and R³ are independently of each other (C₁-C₆)alkyl.

In some embodiments, the invention relates to a composition for improving blood glucose control comprising an effective amount of one or more compounds of Formula I, wherein R¹ and R² are independently of each other (C₁-C₄)alkoxy and R³ is (C₁-C₄)alkyl. In some embodiments, the invention relates to a composition for improving blood glucose control comprising an effective amount of one or more compounds of Formula I, wherein R¹ and R² are independently of each other (C₁-C₄)alkoxy and R³ is hydrogen. In other embodiments, the invention relates to a composition comprising an effective amount of one or more compounds of Formula I, wherein R¹, R² and R³ are independently of each other (C₁-C₄)alkyl or hydrogen. In other embodiments, the invention relates to a composition comprising an effective amount of one or more compounds of Formula I, wherein R¹, R² and R³ are independently of each other (C₁-C₄)alkyl.

In some embodiments, the invention relates to a composition for improving blood glucose control comprising an effective amount of one or more compounds of Formula I, wherein R¹ and R² are methoxy and R³ is methyl. In some embodiments, the invention relates to a composition for improving blood glucose control comprising an effective amount of one or more compounds of Formula I, wherein R¹ and R² are methoxy, and R³ is hydrogen. In other embodiments, the invention relates to a composition comprising an effective amount of one or more compounds of Formula I, wherein R¹, R² and R³ are independently of each other methyl or hydrogen. In other embodiments, the invention relates to a composition comprising an effective amount of one or more compounds of Formula I, wherein R¹, R² and R³ are methyl.

In some embodiments, the invention relates to a composition for improving blood glucose control comprising an effective amount of one or more compounds of Formula I, wherein R¹ and R² are methoxy, R³ is methyl and m is an integer from an integer from 0-4 inclusive. In some embodiments, the invention relates to a composition for improving blood glucose control comprising an effective amount of one or more compounds of Formula I, wherein R¹ and R² are methoxy, R³ is hydrogen and m is an integer from 0-4 inclusive. In other embodiments, the invention relates to a composition comprising an effective amount of one or more compounds of Formula I, wherein R¹, R² and R³ are independently of each other methyl or hydrogen, and m is an integer from 0-4 inclusive. In other embodiments, the invention relates to a composition comprising an effective amount of one or more compounds of Formula I, wherein R¹, R² and R³ are methyl and m is an integer from 0-4 inclusive.

In some embodiments, the invention relates to a composition for improving blood glucose control comprising an effective amount of one or more compounds of Formula I, where the bond indicated by a dashed line is a double bond in every occurrence. In some embodiments, the invention relates to a composition for improving blood glucose control comprising an effective amount of one or more compounds of Formula I, where the bond indicated by a dashed line is a single bond in every occurrence.

In some embodiments, the invention relates to a composition for improving blood glucose control, comprising an effective amount of a compound of Formula I wherein m is the integer zero. In some embodiments, the invention relates to a composition for improving blood glucose control, comprising an effective amount of a compound of Formula I wherein m is the integer one. In some embodiments, the invention relates to a composition for improving blood glucose control, comprising an effective amount of a compound of Formula I wherein m is the integer two. In some embodiments, the invention relates to a composition for improving blood glucose control, comprising an effective amount of a compound of Formula I wherein m is the integer three. In some embodiments, the invention relates to a composition for improving blood glucose control, comprising an effective amount of a compound of Formula I wherein m is the integer four. In some embodiments, the invention relates to a composition for improving blood glucose control, comprising an effective amount of a compound of Formula I wherein m is the integer five. In some embodiments, the invention relates to a composition for improving blood glucose control, comprising an effective amount of a compound of Formula I wherein m is the integer six. In some embodiments, the invention relates to a composition for improving blood glucose control, comprising an effective amount of a compound of Formula I wherein m is the integer seven. In some embodiments, the invention relates to a composition for improving blood glucose control, comprising an effective amount of a compound of Formula I wherein m is the integer eight. In some embodiments, the invention relates to a composition for improving blood glucose control, comprising an effective amount of a compound of Formula I wherein m is the integer nine. In some embodiments, the invention relates to a composition for improving blood glucose control, comprising an effective amount of a compound of Formula I wherein m is the integer ten. In some embodiments, the invention relates to a composition for improving blood glucose control, comprising an effective amount of a compound of Formula I wherein m is the integer eleven. In some embodiments, the invention relates to a composition for improving blood glucose control comprising an effective amount of a compound of Formula I wherein m is the integer twelve. In any of the above embodiments, the bonds indicated by a dashed line in Formula I can be, independently of the other bonds, a double bond or a single bond. In any of the above embodiments, the bonds indicated by a dashed line in Formula I can be a double bond in every occurrence. In any of the above embodiments, the bonds indicated by a dashed line in Formula I can be a single bond in every occurrence.

In some embodiments, the invention relates to a composition for improving blood glucose control, comprising an effective amount of a compound of Formula I, wherein the compound is selected from alpha-tocotrienol quinone, beta-tocotrienol quinone, gamma-tocotrienol quinone or delta-tocotrienol quinone or any stereoisomer or mixture of stereoisomers thereof. In some embodiments, the invention relates to a composition for improving blood glucose control, comprising a mixture of two or more tocotrienol quinones selected from alpha-tocotrienol quinone, beta-tocotrienol quinone, gamma-tocotrienol quinone and delta-tocotrienol quinone or any stereoisomer or mixture of stereoisomers thereof.

In some embodiments, the invention relates to a composition for improving blood glucose control in a subject with abnormal glucose tolerance and metabolism, comprising alpha-tocotrienol quinone and, optionally, a pharmaceutically acceptable carrier, diluent or excipient. In some embodiments, the invention relates to a composition for improving blood glucose control in a subject with abnormal glucose tolerance and metabolism, comprising beta-tocotrienol quinone and, optionally, a pharmaceutically acceptable carrier, diluent or excipient. In some embodiments, the invention relates to a composition for improving blood glucose control in a subject with abnormal glucose tolerance and metabolism, comprising gamma-tocotrienol quinone and, optionally, a pharmaceutically acceptable carrier, diluent or excipient. In some embodiments, the invention relates to a composition for improving blood glucose control in a subject with abnormal glucose tolerance and metabolism, comprising delta-tocotrienol quinone and, optionally, a pharmaceutically acceptable carrier, diluent or excipient.

The present invention further relates to a composition comprising an effective amount of one or more compounds of Formula I or mixtures thereof and, optionally, a pharmaceutically acceptable carrier, diluent or excipient, for preventing, reducing, ameliorating or treating diabetes, or to stabilize the level of blood sugar, stop the increase in level of blood sugar, reverse the increase in the level of blood sugar, or reduce the level of blood sugar in a subject with abnormal glucose tolerance and metabolism in need of such treatment, where the one or more compounds of Formula I have the following structure:

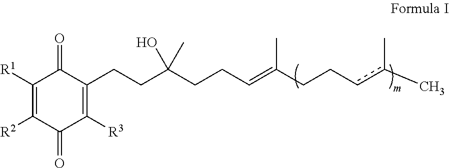

wherein,
each bond indicated by a dashed line can independently be double or single;
R¹, R² and R³ are independently of each other hydrogen, (C₁-C₆)alkyl or (C₁-C₆)alkoxy; and
m is an integer of 0 to 12 inclusive;
or any stereoisomer, mixture of stereoisomers, prodrug, metabolite, salt, crystalline form, non-crystalline form, hydrate or solvate thereof.

In some embodiments, the composition for preventing, reducing, ameliorating or treating diabetes, or to stabilize the level of blood sugar, stop the increase in level of blood sugar, reverse the increase in the level of blood sugar, or reduce the level of blood sugar is administered via oral, gastrointestinal, rectal, parenteral, local, inhalant or enteral route. In other embodiments, the composition for preventing, reducing, ameliorating or treating diabetes, or to stabilize the level of blood sugar, stop the increase in level of blood sugar, reverse the increase in the level of blood sugar, or reduce the level of blood sugar is an oral composition.

In some embodiments, the invention relates to a composition for preventing, reducing, ameliorating or treating diabetes, or to stabilize the level of blood sugar, stop the increase in level of blood sugar, reverse the increase in the level of blood sugar, or reduce the level of blood sugar comprising an effective amount of one or more compounds of Formula I, wherein R¹ and R² are independently of each other (C₁-C₆)alkoxy and R³ is (C₁-C₆)alkyl. In some embodiments, the invention relates to a composition for preventing, reducing, ameliorating or treating diabetes, or to stabilize the level of blood sugar, stop the increase in level of blood sugar, reverse the increase in the level of blood sugar, or reduce the level of blood sugar comprising an effective amount of one or more compounds of Formula I, wherein R¹ and R² are independently of each other (C₁-C₆)alkoxy and R³ is hydrogen. In other embodiments, the invention relates to a composition for preventing, reducing, ameliorating or treating diabetes, or to stabilize the level of blood sugar, stop the increase in level of blood sugar, reverse the increase in the level of blood sugar, or reduce the level of blood sugar comprising an effective amount of one or more compounds of Formula I, wherein R¹, R² and R³ are independently of each other (C₁-C₆)alkyl or hydrogen. In other embodiments, the invention relates to a composition comprising an effective amount of one or more compounds of Formula I, wherein R¹, R² and R³ are independently of each other (C₁-C₆)alkyl.

In some embodiments, the invention relates to a composition for preventing, reducing, ameliorating or treating diabetes, or to stabilize the level of blood sugar, stop the increase in level of blood sugar, reverse the increase in the level of blood sugar, or reduce the level of blood sugar comprising an effective amount of one or more compounds of Formula I, wherein R¹ and R² are independently of each other (C₁-C₄)alkoxy and R³ is (C₁-C₄)alkyl. In some embodiments, the invention relates to a composition for preventing, reducing, ameliorating or treating diabetes, or to stabilize the level of blood sugar, stop the increase in level of blood sugar, reverse the increase in the level of blood sugar, or reduce the level of blood sugar comprising an effective amount of one or more compounds of Formula I, wherein R¹ and R² are independently of each other (C₁-C₄)alkoxy and R³ is hydrogen. In other embodiments, the invention relates to a composition for preventing, reducing, ameliorating or treating diabetes, or to stabilize the level of blood sugar, stop the increase in level of blood sugar, reverse the increase in the level of blood sugar, or reduce the level of blood sugar comprising an effective amount of one or more compounds of Formula I, wherein R¹, R² and R³ are independently of each other (C₁-C₄)alkyl or hydrogen. In other embodiments, the invention relates to a composition for preventing, reducing, ameliorating or treating diabetes, or to stabilize the level of blood sugar, stop the increase in level of blood sugar, reverse the increase in the level of blood sugar, or reduce the level of blood sugar comprising an effective amount of one or more compounds of Formula I, wherein R¹, R² and R³ are independently of each other (C₁-C₄)alkyl.

In some embodiments, the invention relates to a composition for preventing, reducing, ameliorating or treating diabetes, or to stabilize the level of blood sugar, stop the increase in level of blood sugar, reverse the increase in the level of blood sugar, or reduce the level of blood sugar comprising an effective amount of one or more compounds of Formula I, wherein R¹ and R² are methoxy and R³ is methyl. In some embodiments, the invention relates to a composition for preventing, reducing, ameliorating or treating diabetes, or to stabilize the level of blood sugar, stop the increase in level of blood sugar, reverse the increase in the level of blood sugar, or reduce the level of blood sugar comprising an effective amount of one or more compounds of Formula I, wherein R¹ and R² are methoxy and R³ is hydrogen. In other embodiments, the invention relates to a composition for preventing, reducing, ameliorating or treating diabetes, or to stabilize the level of blood sugar, stop the increase in level of blood sugar, reverse the increase in the level of blood sugar, or reduce the level of blood sugar comprising an effective amount of one or more compounds of Formula I, wherein R¹, R² and R³ are independently of each other methyl or hydrogen. In other embodiments, the invention relates to a composition for preventing, reducing, ameliorating or treating diabetes, or to stabilize the level of blood sugar, stop the increase in level of blood sugar, reverse the increase in the level of blood sugar, or reduce the level of blood sugar comprising an effective amount of one or more compounds of Formula I, wherein R¹, R² and R³ are methyl.

In some embodiments, the invention relates to a composition for preventing, reducing, ameliorating or treating diabetes, or to stabilize the level of blood sugar, stop the increase in level of blood sugar, reverse the increase in the level of blood sugar, or reduce the level of blood sugar comprising an effective amount of one or more compounds of Formula I, wherein R¹ and R² are methoxy, R³ is methyl and m is an integer from 0-4 inclusive. In some embodiments, the invention relates to a composition for preventing, reducing, ameliorating or treating diabetes, or to stabilize the level of blood sugar, stop the increase in level of blood sugar, reverse the increase in the level of blood sugar, or reduce the level of blood sugar comprising an effective amount of one or more compounds of Formula I, wherein R¹ and R² are methoxy, R³ is hydrogen, and m is an integer from 0-4 inclusive. In other embodiments, the invention relates to a composition for preventing, reducing, ameliorating or treating diabetes, or to stabilize the level of blood sugar, stop the increase in level of blood sugar, reverse the increase in the level of blood sugar, or reduce the level of blood sugar comprising an effective amount of one or more compounds of Formula I, wherein R¹, R² and R³ are independently of each other methyl or hydrogen and m is an integer from 0-4 inclusive. In other embodiments, the invention relates to a composition for preventing, reducing, ameliorating or treating diabetes, or to stabilize the level of blood sugar, stop the increase in level of blood sugar, reverse the increase in the level of blood sugar, or reduce the level of blood sugar comprising an effective amount of one or more compounds of Formula I, wherein R¹, R² and R³ are methyl and m is an integer from 0-4 inclusive.

In some embodiments, the invention relates to a composition for preventing, reducing, ameliorating or treating diabetes, or to stabilize the level of blood sugar, stop the increase in level of blood sugar, reverse the increase in the level of blood sugar, or reduce the level of blood sugar comprising an effective amount of one or more compounds of Formula I, where the bond indicated by a dashed line is a double bond in every occurrence. In some embodiments, the invention relates to a composition for preventing, reducing, ameliorating or treating diabetes, or to stabilize the level of blood sugar, stop the increase in level of blood sugar, reverse the increase in the level of blood sugar, or reduce the level of blood sugar comprising an effective amount of one or more compounds of Formula I, where the bond indicated by a dashed line is a single bond in every occurrence.

In some embodiments, the invention relates to a composition for preventing, reducing, ameliorating or treating diabetes, or to stabilize the level of blood sugar, stop the increase in level of blood sugar, reverse the increase in the level of blood sugar, or reduce the level of blood sugar, comprising an effective amount of a compound of Formula I wherein m is the integer zero. In some embodiments, the invention relates to a composition for preventing, reducing, ameliorating or treating diabetes, or to stabilize the level of blood sugar, stop the increase in level of blood sugar, reverse the increase in the level of blood sugar, or reduce the level of blood sugar, comprising an effective amount of a compound of Formula I wherein m is the integer one. In some embodiments, the invention relates to a composition for preventing, reducing, ameliorating or treating diabetes, or to stabilize the level of blood sugar, stop the increase in level of blood sugar, reverse the increase in the level of blood sugar, or reduce the level of blood sugar, comprising an effective amount of a compound of Formula I wherein m is the integer two. In some embodiments, the invention relates to a composition for preventing, reducing, ameliorating or treating diabetes, or to stabilize the level of blood sugar, stop the increase in level of blood sugar, reverse the increase in the level of blood sugar, or reduce the level of blood sugar, comprising an effective amount of a compound of Formula I wherein m is the integer three. In some embodiments, the invention relates to a composition for preventing, reducing, ameliorating or treating diabetes, or to stabilize the level of blood sugar, stop the increase in level of blood sugar, reverse the increase in the level of blood sugar, or reduce the level of blood sugar, comprising an effective amount of a compound of Formula I wherein m is the integer four. In some embodiments, the invention relates to a composition for preventing, reducing, ameliorating or treating diabetes, or to stabilize the level of blood sugar, stop the increase in level of blood sugar, reverse the increase in the level of blood sugar, or reduce the level of blood sugar, comprising an effective amount of a compound of Formula I wherein m is the integer five. In some embodiments, the invention relates to a composition for preventing, reducing, ameliorating or treating diabetes, or to stabilize the level of blood sugar, stop the increase in level of blood sugar, reverse the increase in the level of blood sugar, or reduce the level of blood sugar, comprising an effective amount of a compound of Formula I wherein m is the integer six. In some embodiments, the invention relates to a composition for preventing, reducing, ameliorating or treating diabetes, or to stabilize the level of blood sugar, stop the increase in level of blood sugar, reverse the increase in the level of blood sugar, or reduce the level of blood sugar, comprising an effective amount of a compound of Formula I wherein m is the integer seven. In some embodiments, the invention relates to a composition for preventing, reducing, ameliorating or treating diabetes, or to stabilize the level of blood sugar, stop the increase in level of blood sugar, reverse the increase in the level of blood sugar, or reduce the level of blood sugar comprising an effective amount of a compound of Formula I wherein m is the integer eight. In some embodiments, the invention relates to a composition for preventing, reducing, ameliorating or treating diabetes, or to stabilize the level of blood sugar, stop the increase in level of blood sugar, reverse the increase in the level of blood sugar, or reduce the level of blood sugar, comprising an effective amount of a compound of Formula I wherein m is the integer nine. In some embodiments, the invention relates to a composition for preventing, reducing, ameliorating or treating diabetes, or to stabilize the level of blood sugar, stop the increase in level of blood sugar, reverse the increase in the level of blood sugar, or reduce the level of blood sugar, comprising an effective amount of a compound of Formula I wherein m is the integer ten. In some embodiments, the invention relates to a composition for preventing, reducing, ameliorating or treating diabetes, or to stabilize the level of blood sugar, stop the increase in level of blood sugar, reverse the increase in the level of blood sugar, or reduce the level of blood sugar, comprising an effective amount of a compound of Formula I wherein m is the integer eleven. In some embodiments, the invention relates to a composition for preventing, reducing, ameliorating or treating diabetes, or to stabilize the level of blood sugar, stop the increase in level of blood sugar, reverse the increase in the level of blood sugar, or reduce the level of blood sugar, comprising an effective amount of a compound of Formula I wherein m is the integer twelve. In any of the above embodiments, the bonds indicated by a dashed line in Formula I can be, independently of the other bonds, a double bond or a single bond. In any of the above embodiments, the bonds indicated by a dashed line in Formula I can be a double bond in every occurrence. In any of the above embodiments, the bonds indicated by a dashed line in Formula I can be a single bond in every occurrence.

In some embodiments, the invention relates to a composition for preventing, reducing, ameliorating or treating diabetes, or to stabilize the level of blood sugar, stop the increase in level of blood sugar, reverse the increase in the level of blood sugar, or reduce the level of blood sugar in a subject with abnormal glucose tolerance and metabolism, comprising an effective amount of a compound of Formula I, wherein the compound is selected from alpha-tocotrienol quinone, beta-tocotrienol quinone, gamma-tocotrienol quinone or delta-tocotrienol quinone or any stereoisomer or mixture of stereoisomers thereof. In some embodiments, the invention relates to a composition for preventing, reducing, ameliorating or treating diabetes, or to stabilize the level of blood sugar, stop the increase in level of blood sugar, reverse the increase in the level of blood sugar, or reduce the level of blood sugar in a subject with abnormal glucose tolerance and metabolism, comprising a mixture of two or more tocotrienol quinones selected from alpha-tocotrienol quinone, beta-tocotrienol quinone, gamma-tocotrienol quinone and delta-tocotrienol quinone or any stereoisomer or mixture of stereoisomers thereof.

In some embodiments, the invention relates to a composition for preventing, reducing, ameliorating or treating diabetes, or to stabilize the level of blood sugar, stop the increase in level of blood sugar, reverse the increase in the level of blood sugar, or reduce the level of blood sugar in a subject with abnormal glucose tolerance and metabolism, comprising alpha-tocotrienol quinone and, optionally, a pharmaceutically acceptable carrier, diluent or excipient. In some embodiments, the invention relates to a composition for preventing, reducing, ameliorating or treating diabetes, or to stabilize the level of blood sugar, stop the increase in level of blood sugar, reverse the increase in the level of blood sugar, or reduce the level of blood sugar in a subject with abnormal glucose tolerance and metabolism, comprising beta-tocotrienol quinone and, optionally, a pharmaceutically acceptable carrier, diluent or excipient. In some embodiments, the invention relates to a composition for preventing, reducing, ameliorating or treating diabetes, or to stabilize the level of blood sugar, stop the increase in level of blood sugar, reverse the increase in the level of blood sugar, or reduce the level of blood sugar in a subject with abnormal glucose tolerance and metabolism, comprising gamma-tocotrienol quinone and, optionally, a pharmaceutically acceptable carrier, diluent or excipient. In some embodiments, the invention relates to a composition for preventing, reducing, ameliorating or treating diabetes, or to stabilize the level of blood sugar, stop the increase in level of blood sugar, reverse the increase in the level of blood sugar, or reduce the level of blood sugar in a subject with abnormal glucose tolerance and metabolism, comprising delta-tocotrienol quinone and, optionally, a pharmaceutically acceptable carrier, diluent or excipient.

The present invention further relates to a composition comprising an effective amount of one or more compounds of Formula I or mixtures thereof and, optionally, a pharmaceutically acceptable carrier, diluent or excipient, for preventing or treating a condition requiring increasing insulin sensitivity or reducing insulin resistance in a subject with abnormal glucose tolerance and metabolism in need of such treatment, where the one or more compounds of Formula I have the following structure:

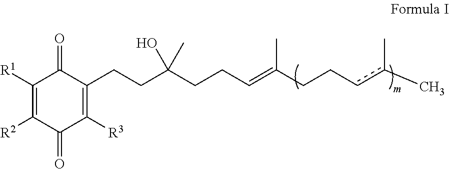

wherein,
each bond indicated by a dashed line can independently be double or single;
R¹, R² and R³ are independently of each other hydrogen, (C₁-C₆)alkyl or (C₁-C₆)alkoxy; and
m is an integer of 0 to 12 inclusive;
or any stereoisomer, mixture of stereoisomers, prodrug, metabolite, salt, crystalline form, non-crystalline form, hydrate or solvate thereof.

In some embodiments, the composition for preventing or treating a condition requiring increasing insulin sensitivity or reducing insulin resistance in a subject with abnormal glucose tolerance and metabolism in need of such treatment, is administered by oral, gastrointestinal, rectal, parenteral, local, inhalant or enteral route. In other embodiments, the composition for preventing or treating a condition requiring increasing insulin sensitivity or reducing insulin resistance in a subject with abnormal glucose tolerance and metabolism in need of such treatment is an oral composition.

In some embodiments, the invention relates to a composition for preventing or treating a condition requiring increasing insulin sensitivity or reducing insulin resistance in a subject with abnormal glucose tolerance and metabolism in need of such treatment, comprising an effective amount of one or more compounds of Formula I, wherein R¹ and R² are independently of each other (C₁-C₆)alkoxy and R³ is (C₁-C₆)alkyl. In some embodiments, the invention relates to a composition for preventing or treating a condition requiring increasing insulin sensitivity or reducing insulin resistance in a subject with abnormal glucose tolerance and metabolism in need of such treatment, comprising an effective amount of one or more compounds of Formula I, wherein R¹ and R² are independently of each other (C₁-C₆)alkoxy and R³ is hydrogen. In other embodiments, the invention relates to a composition for preventing or treating a condition requiring increasing insulin sensitivity or reducing insulin resistance in a subject with abnormal glucose tolerance and metabolism in need of such treatment, comprising an effective amount of one or more compounds of Formula I, wherein R¹, R² and R³ are independently of each other (C₁-C₆)alkyl or hydrogen. In other embodiments, the invention relates to a composition for preventing or treating a condition requiring increasing insulin sensitivity or reducing insulin resistance in a subject with abnormal glucose tolerance and metabolism in need of such treatment, comprising an effective amount of one or more compounds of Formula I, wherein R¹, R² and R³ are independently of each other (C₁-C₆)alkyl.

In some embodiments, the invention relates to a composition for preventing or treating a condition requiring increasing insulin sensitivity or reducing insulin resistance in a subject with abnormal glucose tolerance and metabolism in need of such treatment, comprising an effective amount of one or more compounds of Formula I, wherein R¹ and R² are independently of each other (C₁-C₄)alkoxy and R³ is (C₁-C₄)alkyl. In some embodiments, the invention relates to a composition for preventing or treating a condition requiring increasing insulin sensitivity or reducing insulin resistance in a subject with abnormal glucose tolerance and metabolism in need of such treatment, comprising an effective amount of one or more compounds of Formula I, wherein R¹ and R² are independently of each other (C₁-C₄)alkoxy and R³ is hydrogen. In other embodiments, the invention relates to a composition for preventing or treating a condition requiring increasing insulin sensitivity or reducing insulin resistance in a subject with abnormal glucose tolerance and metabolism in need of such treatment, comprising an effective amount of one or more compounds of Formula I, wherein R¹, R² and R³ are independently of each other (C₁-C₄)alkyl or hydrogen. In other embodiments, the invention relates to a composition for preventing or treating a condition requiring increasing insulin sensitivity or reducing insulin resistance in a subject with abnormal glucose tolerance and metabolism in need of such treatment, comprising an effective amount of one or more compounds of Formula I, wherein R¹, R² and R³ are independently of each other (C₁-C₄)alkyl.

In some embodiments, the invention relates to a composition for preventing or treating a condition requiring increasing insulin sensitivity or reducing insulin resistance in a subject with abnormal glucose tolerance and metabolism in need of such treatment, comprising an effective amount of one or more compounds of Formula I, wherein R¹ and R² are methoxy and R³ is methyl. In some embodiments, the invention relates to a composition for preventing or treating a condition requiring increasing insulin sensitivity or reducing insulin resistance in a subject with abnormal glucose tolerance and metabolism in need of such treatment, comprising an effective amount of one or more compounds of Formula I, wherein R¹ and R² are methoxy and R³ is hydrogen. In other embodiments, the invention relates to a composition for preventing or treating a condition requiring increasing insulin sensitivity or reducing insulin resistance in a subject with abnormal glucose tolerance and metabolism in need of such treatment, comprising an effective amount of one or more compounds of Formula I, wherein R¹, R² and R³ are independently of each other methyl or hydrogen In other embodiments, the invention relates to a composition for preventing or treating a condition requiring increasing insulin sensitivity or reducing insulin resistance in a subject with abnormal glucose tolerance and metabolism in need of such treatment, comprising an effective amount of one or more compounds of Formula I, wherein R¹, R² and R³ are methyl.

In some embodiments, the invention relates to a composition for preventing or treating a condition requiring increasing insulin sensitivity or reducing insulin resistance in a subject with abnormal glucose tolerance and metabolism in need of such treatment, comprising an effective amount of one or more compounds of Formula I, wherein R¹ and R² are methoxy, R³ is methyl and m is an integer from 0-4 inclusive. In some embodiments, the invention relates to a composition for preventing or treating a condition requiring increasing insulin sensitivity or reducing insulin resistance in a subject with abnormal glucose tolerance and metabolism in need of such treatment, comprising an effective amount of one or more compounds of Formula I, wherein R¹ and R² are methoxy, R³ is hydrogen and m is an integer from 0-4 inclusive. In other embodiments, the invention relates to a composition for preventing or treating a condition requiring increasing insulin sensitivity or reducing insulin resistance in a subject with abnormal glucose tolerance and metabolism in need of such treatment, comprising an effective amount of one or more compounds of Formula I, wherein R¹, R² and R³ are independently of each other methyl or hydrogen, and m is an integer from 0-4 inclusive. In other embodiments, the invention relates to a composition for preventing or treating a condition requiring increasing insulin sensitivity or reducing insulin resistance in a subject with abnormal glucose tolerance and metabolism in need of such treatment, comprising an effective amount of one or more compounds of Formula I, wherein R¹, R² and R³ are methyl and m is an integer from 0-4 inclusive.

In some embodiments, the invention relates to a composition for preventing or treating a condition requiring increasing insulin sensitivity or reducing insulin resistance in a subject with abnormal glucose tolerance and metabolism in need of such treatment, comprising an effective amount of one or more compounds of Formula I, where the bond indicated by a dashed line is a double bond in every occurrence. In some embodiments, the invention relates to a composition for preventing or treating a condition requiring increasing insulin sensitivity or reducing insulin resistance in a subject with abnormal glucose tolerance and metabolism in need of such treatment, comprising an effective amount of one or more compounds of Formula I, where the bond indicated by a dashed line is a single bond in every occurrence.

In some embodiments, the invention relates to a composition for preventing or treating a condition requiring increasing insulin sensitivity or reducing insulin resistance comprising an effective amount of a compound of Formula I wherein m is the integer zero. In some embodiments, the invention relates to a composition for preventing or treating a condition requiring increasing insulin sensitivity or reducing insulin resistance comprising an effective amount of a compound of Formula I wherein m is the integer one. In some embodiments, the invention relates to a composition for preventing or treating a condition requiring increasing insulin sensitivity or reducing insulin resistance comprising an effective amount of a compound of Formula I wherein m is the integer two. In some embodiments, the invention relates to a composition for preventing or treating a condition requiring increasing insulin sensitivity or reducing insulin resistance comprising an effective amount of a compound of Formula I wherein m is the integer three. In some embodiments, the invention relates to a composition for preventing or treating a condition requiring increasing insulin sensitivity or reducing insulin resistance comprising an effective amount of a compound of Formula I wherein m is the integer four. In some embodiments, the invention relates to a composition for preventing or treating a condition requiring increasing insulin sensitivity or reducing insulin resistance comprising an effective amount of a compound of Formula I wherein m is the integer five. In some embodiments, the invention relates to a composition for preventing or treating a condition requiring increasing insulin sensitivity or reducing insulin resistance comprising an effective amount of a compound of Formula I wherein m is the integer six. In some embodiments, the invention relates to a composition for preventing or treating a condition requiring increasing insulin sensitivity or reducing insulin resistance comprising an effective amount of a compound of Formula I wherein m is the integer seven. In some embodiments, the invention relates to a composition for preventing or treating a condition requiring increasing insulin sensitivity or reducing insulin resistance comprising an effective amount of a compound of Formula I wherein m is the integer eight. In some embodiments, the invention relates to a composition for preventing or treating a condition requiring increasing insulin sensitivity or reducing insulin resistance comprising an effective amount of a compound of Formula I wherein m is the integer nine. In some embodiments, the invention relates to a composition for preventing or treating a condition requiring increasing insulin sensitivity or reducing insulin resistance comprising an effective amount of a compound of Formula I wherein m is the integer ten. In some embodiments, the invention relates to a composition for preventing or treating a condition requiring increasing insulin sensitivity or reducing insulin resistance comprising an effective amount of a compound of Formula I wherein m is the integer eleven. In some embodiments, the invention relates to a composition for preventing or treating a condition requiring increasing insulin sensitivity or reducing insulin resistance comprising an effective amount of a compound of Formula I wherein m is the integer twelve. In any of the above embodiments, the bonds indicated by a dashed line in Formula I can be, independently of the other bonds, a double bond or a single bond. In any of the above embodiments, the bonds indicated by a dashed line in Formula I can be a double bond in every occurrence. In any of the above embodiments, the bonds indicated by a dashed line in Formula I can be a single bond in every occurrence.

In some embodiments, the invention relates to a composition for preventing or treating a condition requiring increasing insulin sensitivity or reducing insulin resistance in a subject with abnormal glucose tolerance and metabolism, comprising an effective amount of a compound of Formula I, wherein the compound is selected from alpha-tocotrienol quinone, beta-tocotrienol quinone, gamma-tocotrienol quinone or delta-tocotrienol quinone or any stereoisomer or mixture of stereoisomers thereof. In some embodiments, the invention relates to a composition for preventing or treating a condition requiring increasing insulin sensitivity or reducing insulin resistance in a subject with abnormal glucose tolerance and metabolism, comprising a mixture of two or more tocotrienol quinones selected from alpha-tocotrienol quinone, beta-tocotrienol quinone, gamma-tocotrienol quinone and delta-tocotrienol quinone or any stereoisomer or mixture of stereoisomers thereof.

In some embodiments, the invention relates to a composition for preventing or treating a condition requiring increasing insulin sensitivity or reducing insulin resistance in a subject with abnormal glucose tolerance and metabolism, comprising alpha-tocotrienol quinone and, optionally, a pharmaceutically acceptable carrier, diluent or excipient. In some embodiments, the invention relates to a composition for preventing or treating a condition requiring increasing insulin sensitivity or reducing insulin resistance in a subject with abnormal glucose tolerance and metabolism, comprising beta-tocotrienol quinone and, optionally, a pharmaceutically acceptable carrier, diluent or excipient. In some embodiments, the invention relates to a composition for preventing or treating a condition requiring increasing insulin sensitivity or reducing insulin resistance in a subject with abnormal glucose tolerance and metabolism, comprising gamma-tocotrienol quinone and, optionally, a pharmaceutically acceptable carrier, diluent or excipient. In some embodiments, the invention relates to a composition for preventing or treating a condition requiring increasing insulin sensitivity or reducing insulin resistance in a subject with abnormal glucose tolerance and metabolism, comprising delta-tocotrienol quinone and, optionally, a pharmaceutically acceptable carrier, diluent or excipient.

The present invention further relates to a method for improving blood glucose control in a subject with abnormal glucose tolerance and metabolism in need of such treatment which comprises administering an effective amount of one or more compounds of Formula I or mixtures thereof and, optionally, a pharmaceutically acceptable carrier, diluent or excipient, where the one or more compounds of Formula I have the following structure:

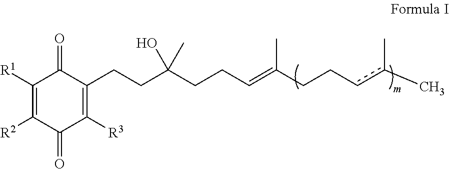

wherein,
each bond indicated by a dashed line can independently be double or single;
R¹, R² and R³ are independently of each other hydrogen, (C₁-C₆)alkyl or (C₁-C₆)alkoxy; and
m is an integer of 0 to 12 inclusive;
or any stereoisomer, mixture of stereoisomers, prodrug, metabolite, salt, crystalline form, non-crystalline form, hydrate or solvate thereof.

In some embodiments, the method for improving blood glucose control comprises administering an effective amount of a composition via oral, gastrointestinal, rectal, parenteral, local, inhalant or enteral route. In other embodiments, the method for improving blood glucose control comprises administering an oral composition.

In some embodiments, the invention relates to a method for improving blood glucose control comprising administering a composition comprising an effective amount of one or more compounds of Formula I, wherein R¹ and R² are independently of each other (C₁-C₆)alkoxy and R³ is (C₁-C₆)alkyl. In some embodiments, the invention relates to a method for improving blood glucose control comprising administering a composition comprising an effective amount of one or more compounds of Formula I, wherein R¹ and R² are independently of each other (C₁-C₆)alkoxy and R³ is hydrogen. In other embodiments, the invention relates to a method for improving blood glucose control comprising administering an effective amount of one or more compounds of Formula I, wherein R¹, R² and R³ are independently of each other (C₁-C₆)alkyl or hydrogen. In other embodiments, the invention relates to a method for improving blood glucose control comprising administering an effective amount of one or more compounds of Formula I, wherein R¹, R² and R³ are independently of each other (C₁-C₆)alkyl.

In some embodiments, the invention relates to a method for improving blood glucose control comprising administering a composition comprising an effective amount of one or more compounds of Formula I, wherein R¹ and R² are independently of each other (C₁-C₄)alkoxy and R³ is (C₁-C₄)alkyl. In some embodiments, the invention relates to a method for improving blood glucose control comprising administering a composition comprising an effective amount of one or more compounds of Formula I, wherein R¹ and R² are independently of each other (C₁-C₄)alkoxy and R³ is hydrogen. In other embodiments, the invention relates to a method for improving blood glucose control comprising administering an effective amount of one or more compounds of Formula I, wherein R¹, R² and R³ are independently of each other (C₁-C₄)alkyl or hydrogen. In other embodiments, the invention relates to a method for improving blood glucose control comprising administering an effective amount of one or more compounds of Formula I, wherein R¹, R² and R³ are independently of each other (C₁-C₄)alkyl.

In some embodiments, the invention relates to a method for improving blood glucose control comprising administering a composition comprising an effective amount of one or more compounds of Formula I, wherein R¹ and R² are methoxy and R³ is methyl. In some embodiments, the invention relates to a method for improving blood glucose control comprising administering a composition comprising an effective amount of one or more compounds of Formula I, wherein R¹ and R² are methoxy and R³ is hydrogen. In other embodiments, the invention relates to a method for improving blood glucose control comprising administering an effective amount of one or more compounds of Formula I, wherein R¹, R² and R³ are independently of each other methyl or hydrogen. In other embodiments, the invention relates to a method for improving blood glucose control comprising administering an effective amount of one or more compounds of Formula I, wherein R¹, R² and R³ are methyl.

In some embodiments, the invention relates to a method for improving blood glucose control comprising administering a composition comprising an effective amount of one or more compounds of Formula I, wherein R¹ and R² are methoxy, R³ is methyl and m is an integer from 0-4 inclusive. In some embodiments, the invention relates to a method for improving blood glucose control comprising administering a composition comprising an effective amount of one or more compounds of Formula I, wherein R¹ and R² are methoxy, R³ is hydrogen and m is an integer from 0-4 inclusive. In other embodiments, the invention relates to a method for improving blood glucose control comprising administering an effective amount of one or more compounds of Formula I, wherein R¹, R² and R³ are independently of each other methyl or hydrogen, and m is an integer from 0-4 inclusive. In other embodiments, the invention relates to a method for improving blood glucose control comprising administering an effective amount of one or more compounds of Formula I, wherein R¹, R² and R³ are methyl and m is an integer from 0-4 inclusive.

In some embodiments, the invention relates to a method for improving blood glucose control comprising administering a composition comprising an effective amount of one or more compounds of Formula I, where the bond indicated by a dashed line is a double bond in every occurrence. In some embodiments, the invention relates to a method for improving blood glucose control comprising administering a composition comprising an effective amount of one or more compounds of Formula I, where the bond indicated by a dashed line is a single bond in every occurrence.

In some embodiments, the invention relates to a method for improving blood glucose control comprising administering a composition comprising an effective amount of a compound of Formula I wherein m is the integer zero. In some embodiments, the invention relates to a method for improving blood glucose control comprising administering a composition comprising an effective amount of a compound of Formula I wherein m is the integer one. In some embodiments, the invention relates to a method for improving blood glucose control comprising administering a composition comprising an effective amount of a compound of Formula I wherein m is the integer two. In some embodiments, the invention relates to a method for improving blood glucose control comprising administering a composition comprising an effective amount of a compound of Formula I wherein m is the integer three. In some embodiments, the invention relates to a method for improving blood glucose control comprising administering a composition comprising an effective amount of a compound of Formula I wherein m is the integer four. In some embodiments, the invention relates to a method for improving blood glucose control comprising administering a composition comprising an effective amount of a compound of Formula I wherein m is the integer five. In some embodiments, the invention relates to a method for improving blood glucose control comprising administering a composition comprising an effective amount of a compound of Formula I wherein m is the integer six. In some embodiments, the invention relates to a method for improving blood glucose control comprising administering a composition comprising an effective amount of a compound of Formula I wherein m is the integer seven. In some embodiments, the invention relates to a method for improving blood glucose control comprising administering a composition comprising an effective amount of a compound of Formula I wherein m is the integer eight. In some embodiments, the invention relates to a method for improving blood glucose control comprising administering a composition comprising an effective amount of a compound of Formula I wherein m is the integer nine. In some embodiments, the invention relates to a method for improving blood glucose control comprising administering a composition comprising an effective amount of a compound of Formula I wherein m is the integer ten. In some embodiments, the invention relates to a method for improving blood glucose control comprising administering a composition comprising an effective amount of a compound of Formula I wherein m is the integer eleven. In some embodiments, the invention relates to a method for improving blood glucose control comprising administering a composition comprising an effective amount of a compound of Formula I wherein m is the integer twelve. In any of the above embodiments, the bonds indicated by a dashed line in Formula I can be, independently of the other bonds, a double bond or a single bond. In any of the above embodiments, the bonds indicated by a dashed line in Formula I can be a double bond in every occurrence. In any of the above embodiments, the bonds indicated by a dashed line in Formula I can be a single bond in every occurrence.

In some embodiments, the invention relates to a method for improving blood glucose control in a subject with abnormal glucose tolerance and metabolism in need of such treatment, comprising administering a composition comprising an effective amount of a compound of Formula I, wherein the compound is selected from alpha-tocotrienol quinone, beta-tocotrienol quinone, gamma-tocotrienol quinone or delta-tocotrienol quinone or any stereoisomer or mixture of stereoisomers thereof. In some embodiments, the invention relates to a method for improving blood glucose control in a subject with abnormal glucose tolerance and metabolism in need of such treatment, comprising administering a composition comprising an effective amount of a mixture of two or more tocotrienol quinones selected from alpha-tocotrienol quinone, beta-tocotrienol quinone, gamma-tocotrienol quinone and delta-tocotrienol quinone or any stereoisomer or mixture of stereoisomers thereof.

In some embodiments, the invention relates to a method for improving blood glucose control in a subject with abnormal glucose tolerance and metabolism, comprising administering a composition comprising alpha-tocotrienol quinone and, optionally, a pharmaceutically acceptable carrier, diluent or excipient. In some embodiments, the invention relates to a method for improving blood glucose control in a subject with abnormal glucose tolerance and metabolism, comprising administering a composition comprising beta-tocotrienol quinone and, optionally, a pharmaceutically acceptable carrier, diluent or excipient. In some embodiments, the invention relates to a method for improving blood glucose control in a subject with abnormal glucose tolerance and metabolism, comprising administering a composition comprising gamma-tocotrienol quinone and, optionally, a pharmaceutically acceptable carrier, diluent or excipient. In some embodiments, the invention relates to a method for improving blood glucose control in a subject with abnormal glucose tolerance and metabolism, comprising administering a composition comprising delta-tocotrienol quinone and, optionally, a pharmaceutically acceptable carrier, diluent or excipient.

The present invention further relates to a method for preventing, reducing, ameliorating or treating diabetes, or to stabilize the level of blood sugar, stop the increase in level of blood sugar, reverse the increase in the level of blood sugar, or reduce the level of blood sugar in a subject with abnormal glucose tolerance and metabolism in need of such treatment, which comprises administering an effective amount of one or more compounds of Formula I or mixtures thereof and, optionally, a pharmaceutically acceptable carrier, diluent or excipient, where the one or more compounds of Formula I have the following structure:

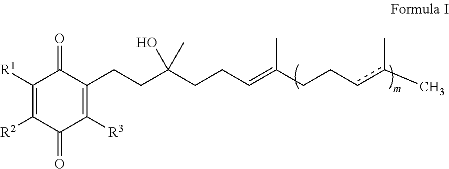

wherein,
each bond indicated by a dashed line can independently be double or single;
R¹, R² and R³ are independently of each other hydrogen, (C₁-C₆)alkyl or (C₁-C₆)alkoxy; and
m is an integer of 0 to 12 inclusive;
or any stereoisomer, mixture of stereoisomers, prodrug, metabolite, salt, crystalline form, non-crystalline form, hydrate or solvate thereof.

In some embodiments, the invention relates to a method for preventing, reducing, ameliorating or treating diabetes, or to stabilize the level of blood sugar, stop the increase in level of blood sugar, reverse the increase in the level of blood sugar, or reduce the level of blood sugar in a subject in need of treatment which comprises administering a composition via oral, gastrointestinal, rectal, parenteral, local, inhalant or enteral route. In other embodiments, the invention relates to a method for preventing, reducing, ameliorating or treating diabetes, or to stabilize the level of blood sugar, stop the increase in level of blood sugar, reverse the increase in the level of blood sugar, or reduce the level of blood sugar in a subject in need of such treatment which comprises administering an oral composition.

In some embodiments, the invention relates to a method for preventing, reducing, ameliorating or treating diabetes, or to stabilize the level of blood sugar, stop the increase in level of blood sugar, reverse the increase in the level of blood sugar, or reduce the level of blood sugar in a subject in need of such treatment which comprises administering a composition comprising an effective amount of one or more compounds of Formula I, wherein R¹ and R² are independently of each other (C₁-C₆)alkoxy and R³ is (C₁-C₆)alkyl. In some embodiments, the invention relates to a method for preventing, reducing, ameliorating or treating diabetes, or to stabilize the level of blood sugar, stop the increase in level of blood sugar, reverse the increase in the level of blood sugar, or reduce the level of blood sugar in a subject in need of such treatment which comprises administering a composition comprising an effective amount of one or more compounds of Formula I, wherein R¹ and R² are independently of each other (C₁-C₆)alkoxy and R³ is hydrogen. In other embodiments, the invention relates to a method for preventing, reducing, ameliorating or treating diabetes, or to stabilize the level of blood sugar, stop the increase in level of blood sugar, reverse the increase in the level of blood sugar, or reduce the level of blood sugar in a subject in need of such control which comprises administering a composition comprising an effective amount of one or more compounds of Formula I, wherein R¹, R² and R³ are independently of each other (C₁-C₆)alkyl or hydrogen. In other embodiments, the invention relates to a method for preventing, reducing, ameliorating or treating diabetes, or to stabilize the level of blood sugar, stop the increase in level of blood sugar, reverse the increase in the level of blood sugar, or reduce the level of blood sugar in a subject in need of such control which comprises administering a composition comprising an effective amount of one or more compounds of Formula I, wherein R¹, R² and R³ are independently of each other (C₁-C₆)alkyl.

In some embodiments, the invention relates to a method for preventing, reducing, ameliorating or treating diabetes, or to stabilize the level of blood sugar, stop the increase in level of blood sugar, reverse the increase in the level of blood sugar, or reduce the level of blood sugar in a subject in need of such treatment which comprises administering a composition comprising an effective amount of one or more compounds of Formula I, wherein R¹ and R² are independently of each other (C₁-C₄)alkoxy and R³ is (C₁-C₄)alkyl. In some embodiments, the invention relates to a method for preventing, reducing, ameliorating or treating diabetes, or to stabilize the level of blood sugar, stop the increase in level of blood sugar, reverse the increase in the level of blood sugar, or reduce the level of blood sugar in a subject in need of such treatment which comprises administering a composition comprising an effective amount of one or more compounds of Formula I, wherein R¹ and R² are independently of each other (C₁-C₄)alkoxy and R³ is hydrogen. In other embodiments, the invention relates to a method for preventing, reducing, ameliorating or treating diabetes, or to stabilize the level of blood sugar, stop the increase in level of blood sugar, reverse the increase in the level of blood sugar, or reduce the level of blood sugar in a subject in need of such control which comprises administering a composition comprising an effective amount of one or more compounds of Formula I, wherein R¹, R² and R³ are independently of each other (C₁-C₄)alkyl or hydrogen. In other embodiments, the invention relates to a method for preventing, reducing, ameliorating or treating diabetes, or to stabilize the level of blood sugar, stop the increase in level of blood sugar, reverse the increase in the level of blood sugar, or reduce the level of blood sugar in a subject in need of such control which comprises administering a composition comprising an effective amount of one or more compounds of Formula I, wherein R¹, R² and R³ are independently of each other (C₁-C₄)alkyl.

In some embodiments, the invention relates to a method for preventing, reducing, ameliorating or treating diabetes, or to stabilize the level of blood sugar, stop the increase in level of blood sugar, reverse the increase in the level of blood sugar, or reduce the level of blood sugar in a subject in need of such treatment which comprises administering a composition comprising an effective amount of one or more compounds of Formula I, wherein R¹ and R² are independently of each other methoxy and R³ is methyl. In some embodiments, the invention relates to a method for preventing, reducing, ameliorating or treating diabetes, or to stabilize the level of blood sugar, stop the increase in level of blood sugar, reverse the increase in the level of blood sugar, or reduce the level of blood sugar in a subject in need of such treatment which comprises administering a composition comprising an effective amount of one or more compounds of Formula I, wherein R¹ and R² are independently of each other methoxy and R³ is hydrogen. In other embodiments, the invention relates to a method for preventing, reducing, ameliorating or treating diabetes, or to stabilize the level of blood sugar, stop the increase in level of blood sugar, reverse the increase in the level of blood sugar, or reduce the level of blood sugar in a subject in need of such control which comprises administering a composition comprising an effective amount of one or more compounds of Formula I, wherein R¹, R² and R³ are methyl or hydrogen. In other embodiments, the invention relates to a method for preventing, reducing, ameliorating or treating diabetes, or to stabilize the level of blood sugar, stop the increase in level of blood sugar, reverse the increase in the level of blood sugar, or reduce the level of blood sugar in a subject in need of such control which comprises administering a composition comprising an effective amount of one or more compounds of Formula I, wherein R¹, R² and R³ are methyl.

In some embodiments, the invention relates to a method for preventing, reducing, ameliorating or treating diabetes, or to stabilize the level of blood sugar, stop the increase in level of blood sugar, reverse the increase in the level of blood sugar, or reduce the level of blood sugar in a subject in need of such treatment which comprises administering a composition comprising an effective amount of one or more compounds of Formula I, wherein R¹ and R² are methoxy, R³ is methyl and m is an integer from 0-4 inclusive. In some embodiments, the invention relates to a method for preventing, reducing, ameliorating or treating diabetes, or to stabilize the level of blood sugar, stop the increase in level of blood sugar, reverse the increase in the level of blood sugar, or reduce the level of blood sugar in a subject in need of such treatment which comprises administering a composition comprising an effective amount of one or more compounds of Formula I, wherein R¹ and R² are independently of each other methoxy, R³ is hydrogen and m is an integer from 0-4 inclusive. In other embodiments, the invention relates to a method for preventing, reducing, ameliorating or treating diabetes, or to stabilize the level of blood sugar, stop the increase in level of blood sugar, reverse the increase in the level of blood sugar, or reduce the level of blood sugar in a subject in need of such control which comprises administering a composition comprising an effective amount of one or more compounds of Formula I, wherein R¹, R² and R³ are methyl or hydrogen, and m is an integer from 0-4 inclusive. In other embodiments, the invention relates to a method for preventing, reducing, ameliorating or treating diabetes, or to stabilize the level of blood sugar, stop the increase in level of blood sugar, reverse the increase in the level of blood sugar, or reduce the level of blood sugar in a subject in need of such control which comprises administering a composition comprising an effective amount of one or more compounds of Formula I, wherein R¹, R² and R³ are methyl and m is an integer from 0-4 inclusive.

In some embodiments, the invention relates to a method for preventing, reducing, ameliorating or treating diabetes, or to stabilize the level of blood sugar, stop the increase in level of blood sugar, reverse the increase in the level of blood sugar, or reduce the level of blood sugar comprising administering an effective amount of one or more compounds of Formula I, where the bond indicated by a dashed line is a double bond in every occurrence. In some embodiments, the invention relates to a method for preventing, reducing, ameliorating or treating diabetes, or to stabilize the level of blood sugar, stop the increase in level of blood sugar, reverse the increase in the level of blood sugar, or reduce the level of blood sugar comprising administering an effective amount of one or more compounds of Formula I, where the bond indicated by a dashed line is a single bond in every occurrence.

In some embodiments, the invention relates to a method for preventing, reducing, ameliorating or treating diabetes, or to stabilize the level of blood sugar, stop the increase in level of blood sugar, reverse the increase in the level of blood sugar, or reduce the level of blood sugar in a subject in need of such treatment which comprises administering a composition comprising an effective amount of a compound of Formula I wherein m is the integer zero. In some embodiments, the invention relates to a method for preventing, reducing, ameliorating or treating diabetes, or to stabilize the level of blood sugar, stop the increase in level of blood sugar, reverse the increase in the level of blood sugar, or reduce the level of blood sugar in a subject in need of such treatment which comprises administering a composition comprising an effective amount of a compound of Formula I wherein m is the integer one. In some embodiments, the invention relates to a method for preventing, reducing, ameliorating or treating diabetes, or to stabilize the level of blood sugar, stop the increase in level of blood sugar, reverse the increase in the level of blood sugar, or reduce the level of blood sugar in a subject in need of such treatment which comprises administering a composition comprising an effective amount of a compound of Formula I wherein m is the integer two. In some embodiments, the invention relates to a method for preventing, reducing, ameliorating or treating diabetes, or to stabilize the level of blood sugar, stop the increase in level of blood sugar, reverse the increase in the level of blood sugar, or reduce the level of blood sugar in a subject in need of such treatment which comprises administering a composition comprising an effective amount of a compound of Formula I wherein m is the integer three. In some embodiments, the invention relates to a method for preventing, reducing, ameliorating or treating diabetes, or to stabilize the level of blood sugar, stop the increase in level of blood sugar, reverse the increase in the level of blood sugar, or reduce the level of blood sugar in a subject in need of such treatment which comprises administering a composition comprising an effective amount of a compound of Formula I wherein m is the integer four. In some embodiments, the invention relates to a method for preventing, reducing, ameliorating or treating diabetes, or to stabilize the level of blood sugar, stop the increase in level of blood sugar, reverse the increase in the level of blood sugar, or reduce the level of blood sugar in a subject in need of such treatment which comprises administering a composition comprising an effective amount of a compound of Formula I wherein m is the integer five. In some embodiments, the invention relates to a method for preventing, reducing, ameliorating or treating diabetes, or to stabilize the level of blood sugar, stop the increase in level of blood sugar, reverse the increase in the level of blood sugar, or reduce the level of blood sugar in a subject in need of such treatment which comprises administering a composition comprising an effective amount of a compound of Formula I wherein m is the integer six. In some embodiments, the invention relates to a method for preventing, reducing, ameliorating or treating diabetes, or to stabilize the level of blood sugar, stop the increase in level of blood sugar, reverse the increase in the level of blood sugar, or reduce the level of blood sugar in a subject in need of such treatment which comprises administering a composition comprising an effective amount of a compound of Formula I wherein m is the integer seven. In some embodiments, the invention relates to a method for preventing, reducing, ameliorating or treating diabetes, or to stabilize the level of blood sugar, stop the increase in level of blood sugar, reverse the increase in the level of blood sugar, or reduce the level of blood sugar in a subject in need of such treatment which comprises administering a composition comprising an effective amount of a compound of Formula I wherein m is the integer eight. In some embodiments, the invention relates to a method for preventing, reducing, ameliorating or treating diabetes, or to stabilize the level of blood sugar, stop the increase in level of blood sugar, reverse the increase in the level of blood sugar, or reduce the level of blood sugar in a subject in need of such treatment which comprises administering a composition comprising an effective amount of a compound of Formula I wherein m is the integer nine. In some embodiments, the invention relates to a method for preventing, reducing, ameliorating or treating diabetes, or to stabilize the level of blood sugar, stop the increase in level of blood sugar, reverse the increase in the level of blood sugar, or reduce the level of blood sugar in a subject in need of such treatment which comprises administering a composition comprising an effective amount of a compound of Formula I wherein m is the integer ten. In some embodiments, the invention relates to a method for preventing, reducing, ameliorating or treating diabetes, or to stabilize the level of blood sugar, stop the increase in level of blood sugar, reverse the increase in the level of blood sugar, or reduce the level of blood sugar in a subject in need of such treatment which comprises administering a composition comprising an effective amount of a compound of Formula I wherein m is the integer eleven. In some embodiments, the invention relates to a method for preventing, reducing, ameliorating or treating diabetes, or to stabilize the level of blood sugar, stop the increase in level of blood sugar, reverse the increase in the level of blood sugar, or reduce the level of blood sugar in a subject in need of such treatment which comprises administering a composition comprising an effective amount of a compound of Formula I wherein m is the integer twelve. In any of the above embodiments, the bonds indicated by a dashed line in Formula I can be, independently of the other bonds, a double bond or a single bond. In any of the above embodiments, the bonds indicated by a dashed line in Formula I can be a double bond in every occurrence. In any of the above embodiments, the bonds indicated by a dashed line in Formula I can be a single bond in every occurrence.

In some embodiments, the invention relates to a method for preventing, reducing, ameliorating or treating diabetes, or to stabilize the level of blood sugar, stop the increase in level of blood sugar, reverse the increase in the level of blood sugar, or reduce the level of blood sugar in a subject with abnormal glucose tolerance and metabolism in need of such treatment which comprises administering a composition comprising an effective amount of a compound of Formula I, wherein the compound is selected from alpha-tocotrienol quinone, beta-tocotrienol quinone, gamma-tocotrienol quinone or delta-tocotrienol quinone or any stereoisomer or mixture of stereoisomers thereof. In some embodiments, the invention relates to a method for preventing, reducing, ameliorating or treating diabetes, or to stabilize the level of blood sugar, stop the increase in level of blood sugar, reverse the increase in the level of blood sugar, or reduce the level of blood sugar in a subject with abnormal glucose tolerance and metabolism in need of such treatment, comprising administering a composition comprising an effective amount of a mixture of two or more tocotrienol quinones selected from alpha-tocotrienol quinone, beta-tocotrienol quinone, gamma-tocotrienol quinone and delta-tocotrienol quinone or any stereoisomer or mixture of stereoisomers thereof.

In some embodiments, the invention relates to a method for preventing, reducing, ameliorating or treating diabetes, or to stabilize the level of blood sugar, stop the increase in level of blood sugar, reverse the increase in the level of blood sugar, or reduce the level of blood sugar in a subject with abnormal glucose tolerance and metabolism in need of such treatment which comprises administering a composition comprising alpha-tocotrienol quinone and, optionally, a pharmaceutically acceptable carrier, diluent or excipient. In some embodiments, the invention relates to a method for preventing, reducing, ameliorating or treating diabetes, or to stabilize the level of blood sugar, stop the increase in level of blood sugar, reverse the increase in the level of blood sugar, or reduce the level of blood sugar in a subject with abnormal glucose tolerance and metabolism in need of such treatment which comprises administering a composition comprising beta-tocotrienol quinone and, optionally, a pharmaceutically acceptable carrier, diluent or excipient. In some embodiments, the invention relates to a method for preventing, reducing, ameliorating or treating diabetes, or to stabilize the level of blood sugar, stop the increase in level of blood sugar, reverse the increase in the level of blood sugar, or reduce the level of blood sugar in a subject with abnormal glucose tolerance and metabolism in need of such treatment which comprises administering a composition comprising gamma-tocotrienol quinone and, optionally, a pharmaceutically acceptable carrier, diluent or excipient. In some embodiments, the invention relates to a method for preventing, reducing, ameliorating or treating diabetes, or to stabilize the level of blood sugar, stop the increase in level of blood sugar, reverse the increase in the level of blood sugar, or reduce the level of blood sugar in a subject with abnormal glucose tolerance and metabolism in need of such treatment which comprises administering a composition comprising delta-tocotrienol quinone and, optionally, a pharmaceutically acceptable carrier, diluent or excipient.

The present invention further relates to a method for preventing or treating a condition requiring increasing insulin sensitivity or reducing insulin resistance in a subject with abnormal glucose tolerance and metabolism in need of such treatment which comprises administering an effective amount of one or more compounds of Formula I or mixtures thereof and, optionally, a pharmaceutically acceptable carrier, diluent or excipient, where the one or more compounds of Formula I have the following structure:

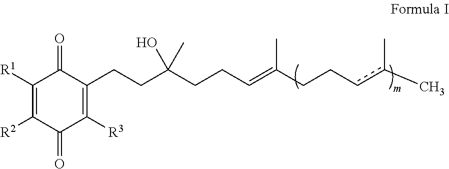

wherein,
each bond indicated by a dashed line can independently be double or single;
R¹, R² and R³ are independently of each other hydrogen, (C₁-C₆)alkyl or (C₁-C₆)alkoxy; and
m is an integer of 0 to 12 inclusive;
or any stereoisomer, mixture of stereoisomers, prodrug, metabolite, salt, crystalline form, non-crystalline form, hydrate or solvate thereof.

In some embodiments, the method for preventing or treating a condition requiring increasing insulin sensitivity or reducing insulin resistance in a subject in need of such control which comprises administering an effective amount of a composition via oral, gastrointestinal, rectal, parenteral, local, inhalant or enteral route. In other embodiments, the method for preventing or treating a condition requiring increasing insulin sensitivity or reducing insulin resistance in a subject with abnormal glucose tolerance and metabolism in need of such treatment, comprises administering an oral composition.

In some embodiments, the invention relates to a method for preventing or treating a condition requiring increasing insulin sensitivity or reducing insulin resistance in a subject in need of such control which comprises administering an effective amount of a composition comprising an effective amount of one or more compounds of Formula I, wherein R¹ and R² are independently of each other (C₁-C₆)alkoxy and R³ is (C₁-C₆) alkyl. In some embodiments, the invention relates to a method for preventing or treating a condition requiring increasing insulin sensitivity or reducing insulin resistance in a subject in need of such control which comprises administering an effective amount of a composition comprising an effective amount of one or more compounds of Formula I, wherein R¹ and R² are independently of each other (C₁-C₆)alkoxy and R³ is hydrogen. In other embodiments, the invention relates to method for preventing or treating a condition requiring increasing insulin sensitivity or reducing insulin resistance in a subject in need of such control which comprises administering an effective amount of a composition comprising an effective amount of one or more compounds of Formula I, wherein R¹, R² and R³ are independently of each other (C₁-C₆)alkyl or hydrogen. In other embodiments, the invention relates to method for preventing or treating a condition requiring increasing insulin sensitivity or reducing insulin resistance in a subject in need of such control which comprises administering an effective amount of a composition comprising an effective amount of one or more compounds of Formula I, wherein R¹, R² and R³ are independently of each other (C₁-C₆)alkyl.

In some embodiments, the invention relates to a method for preventing or treating a condition requiring increasing insulin sensitivity or reducing insulin resistance in a subject in need of such control which comprises administering an effective amount of a composition comprising an effective amount of one or more compounds of Formula I, wherein R¹ and R² are independently of each other (C₁-C₄)alkoxy and R³ is (C₁-C₄) alkyl. In some embodiments, the invention relates to a method for preventing or treating a condition requiring increasing insulin sensitivity or reducing insulin resistance in a subject in need of such control which comprises administering an effective amount of a composition comprising an effective amount of one or more compounds of Formula I, wherein R¹ and R² are independently of each other (C₁-C₄)alkoxy and R³ is hydrogen. In other embodiments, the invention relates to method for preventing or treating a condition requiring increasing insulin sensitivity or reducing insulin resistance in a subject in need of such control which comprises administering an effective amount of a composition comprising an effective amount of one or more compounds of Formula I, wherein R¹, R² and R³ are independently of each other (C₁-C₄)alkyl or hydrogen. In other embodiments, the invention relates to method for preventing or treating a condition requiring increasing insulin sensitivity or reducing insulin resistance in a subject in need of such control which comprises administering an effective amount of a composition comprising an effective amount of one or more compounds of Formula I, wherein R¹, R² and R³ are independently of each other (C₁-C₄)alkyl.

In some embodiments, the invention relates to a method for preventing or treating a condition requiring increasing insulin sensitivity or reducing insulin resistance in a subject in need of such control which comprises administering an effective amount of a composition comprising an effective amount of one or more compounds of Formula I, wherein R¹ and R² are methoxy and R³ is methyl. In some embodiments, the invention relates to a method for preventing or treating a condition requiring increasing insulin sensitivity or reducing insulin resistance in a subject in need of such control which comprises administering an effective amount of a composition comprising an effective amount of one or more compounds of Formula I, wherein R¹ and R² are methoxy, and R³ is hydrogen. In other embodiments, the invention relates to method for preventing or treating a condition requiring increasing insulin sensitivity or reducing insulin resistance in a subject in need of such control which comprises administering an effective amount of a composition comprising an effective amount of one or more compounds of Formula I, wherein R¹, R² and R³ are methyl or hydrogen. In other embodiments, the invention relates to method for preventing or treating a condition requiring increasing insulin sensitivity or reducing insulin resistance in a subject in need of such control which comprises administering an effective amount of a composition comprising an effective amount of one or more compounds of Formula I, wherein R¹, R² and R³ are methyl.

In some embodiments, the invention relates to a method for preventing or treating a condition requiring increasing insulin sensitivity or reducing insulin resistance in a subject in need of such control which comprises administering an effective amount of a composition comprising an effective amount of one or more compounds of Formula I, wherein R¹ and R² are methoxy, R³ is methyl and m is an integer from 0-4 inclusive. In some embodiments, the invention relates to a method for preventing or treating a condition requiring increasing insulin sensitivity or reducing insulin resistance in a subject in need of such control which comprises administering an effective amount of a composition comprising an effective amount of one or more compounds of Formula I, wherein R¹ and R² are methoxy, R³ is hydrogen and m is an integer from 0-4 inclusive. In other embodiments, the invention relates to method for preventing or treating a condition requiring increasing insulin sensitivity or reducing insulin resistance in a subject in need of such control which comprises administering an effective amount of a composition comprising an effective amount of one or more compounds of Formula I, wherein R¹, R² and R³ are methyl or hydrogen, and m is an integer from 0-4 inclusive. In other embodiments, the invention relates to method for preventing or treating a condition requiring increasing insulin sensitivity or reducing insulin resistance in a subject in need of such control which comprises administering an effective amount of a composition comprising an effective amount of one or more compounds of Formula I, wherein R¹, R² and R³ are methyl and m is an integer from 0-4 inclusive.

In some embodiments, the invention relates to a method for preventing or treating a condition requiring increasing insulin sensitivity or reducing insulin resistance in a subject in need of such control which comprises administering an effective amount of a composition comprising an effective amount of one or more compounds of Formula I, where the bond indicated by a dashed line is a double bond in every occurrence. In some embodiments, the invention relates to a method for preventing or treating a condition requiring increasing insulin sensitivity or reducing insulin resistance in a subject in need of such control which comprises administering an effective amount of a composition comprising an effective amount of one or more compounds of Formula I, where the bond indicated by a dashed line is a single bond in every occurrence.

In some embodiments, the invention relates to a method for preventing or treating a condition requiring increasing insulin sensitivity or reducing insulin resistance in a subject with abnormal glucose tolerance and metabolism in need of such treatment which comprises administering a composition comprising an effective amount of a compound of Formula I wherein m is the integer zero. In some embodiments, the invention relates to a method for preventing or treating a condition requiring increasing insulin sensitivity or reducing insulin resistance in a subject with abnormal glucose tolerance and metabolism in need of such treatment which comprises administering a composition comprising an effective amount of a compound of Formula I wherein m is the integer one. In some embodiments, the invention relates to a method for preventing or treating a condition requiring increasing insulin sensitivity or reducing insulin resistance in a subject with abnormal glucose tolerance and metabolism in need of such treatment which comprises administering a composition comprising an effective amount of a compound of Formula I wherein m is the integer two. In some embodiments, the invention relates to a method for preventing or treating a condition requiring increasing insulin sensitivity or reducing insulin resistance in a subject with abnormal glucose tolerance and metabolism in need of such treatment which comprises administering a composition comprising an effective amount of a compound of Formula I wherein m is the integer three. In some embodiments, the invention relates to a method for preventing or treating a condition requiring increasing insulin sensitivity or reducing insulin resistance in a subject with abnormal glucose tolerance and metabolism in need of such treatment which comprises administering a composition comprising an effective amount of a compound of Formula I wherein m is the integer four. In some embodiments, the invention relates to a method for preventing or treating a condition requiring increasing insulin sensitivity or reducing insulin resistance in a subject with abnormal glucose tolerance and metabolism in need of such treatment which comprises administering a composition comprising an effective amount of a compound of Formula I wherein m is the integer five. In some embodiments, the invention relates to a method for preventing or treating a condition requiring increasing insulin sensitivity or reducing insulin resistance in a subject with abnormal glucose tolerance and metabolism in need of such treatment which comprises administering a composition comprising an effective amount of a compound of Formula I wherein m is the integer six. In some embodiments, the invention relates to a method for preventing or treating a condition requiring increasing insulin sensitivity or reducing insulin resistance in a subject with abnormal glucose tolerance and metabolism in need of such treatment which comprises administering a composition comprising an effective amount of a compound of Formula I wherein m is the integer seven. In some embodiments, the invention relates to the method for preventing or treating a condition requiring increasing insulin sensitivity or reducing insulin resistance in a subject with abnormal glucose tolerance and metabolism in need of such treatment which comprises administering a composition comprising an effective amount of a compound of Formula I wherein m is the integer eight. In some embodiments, the invention relates to a method for preventing or treating a condition requiring increasing insulin sensitivity or reducing insulin resistance in a subject with abnormal glucose tolerance and metabolism in need of such treatment which comprises administering a composition comprising an effective amount of a compound of Formula I wherein m is the integer nine. In some embodiments, the invention relates to a method for preventing or treating a condition requiring increasing insulin sensitivity or reducing insulin resistance in a subject with abnormal glucose tolerance and metabolism in need of such treatment which comprises administering a composition comprising an effective amount of a compound of Formula I wherein m is the integer ten. In some embodiments, the invention relates to a method for preventing or treating a condition requiring increasing insulin sensitivity or reducing insulin resistance in a subject with abnormal glucose tolerance and metabolism in need of such treatment which comprises administering a composition comprising an effective amount of a compound of Formula I wherein m is the integer eleven. In some embodiments, the invention relates to a method for preventing or treating a condition requiring increasing insulin sensitivity or reducing insulin resistance in a subject with abnormal glucose tolerance and metabolism in need of such treatment which comprises administering a composition comprising an effective amount of a compound of Formula I wherein m is the integer twelve. In any of the above embodiments, the bonds indicated by a dashed line in Formula I can be, independently of the other bonds, a double bond or a single bond. In any of the above embodiments, the bonds indicated by a dashed line in Formula I can be a double bond in every occurrence. In any of the above embodiments, the bonds indicated by a dashed line in Formula I can be a single bond in every occurrence.

In some embodiments, the invention relates to a method for preventing or treating a condition requiring increasing insulin sensitivity or reducing insulin resistance in a subject with abnormal glucose tolerance and metabolism in need of such treatment which comprises administering an effective amount of a composition comprising a compound of Formula I, wherein the compound is selected from alpha-tocotrienol quinone, beta-tocotrienol quinone, gamma-tocotrienol quinone or delta-tocotrienol quinone or any stereoisomer or mixture of stereoisomers thereof. In some embodiments, the invention relates to a method for preventing or treating a condition requiring increasing insulin sensitivity or reducing insulin resistance in a subject with abnormal glucose tolerance and metabolism in need of such treatment, comprising administering a composition comprising an effective amount of a mixture of two or more tocotrienol quinones selected from alpha-tocotrienol quinone, beta-tocotrienol quinone, gamma-tocotrienol quinone and delta-tocotrienol quinone or any stereoisomer or mixture of stereoisomers thereof.

In some embodiments, the invention relates to a method for preventing or treating a condition requiring increasing insulin sensitivity or reducing insulin resistance in a subject with abnormal glucose tolerance and metabolism in need of such treatment which comprises administering an effective amount of a composition comprising alpha-tocotrienol quinone and, optionally, a pharmaceutically acceptable carrier, diluent or excipient. In some embodiments, the invention relates to a method for preventing or treating a condition requiring increasing insulin sensitivity or reducing insulin resistance in a subject with abnormal glucose tolerance and metabolism in need of such treatment which comprises administering an effective amount of a composition comprising beta-tocotrienol quinone and, optionally, a pharmaceutically acceptable carrier, diluent or excipient. In some embodiments, the invention relates to a method for preventing or treating a condition requiring increasing insulin sensitivity or reducing insulin resistance in a subject with abnormal glucose tolerance and metabolism in need of such treatment which comprises administering an effective amount of a composition comprising gamma-tocotrienol quinone and, optionally, a pharmaceutically acceptable carrier, diluent or excipient. In some embodiments, the invention relates to a method for preventing or treating a condition requiring increasing insulin sensitivity or reducing insulin resistance in a subject with abnormal glucose tolerance and metabolism in need of such treatment which comprises administering an effective amount of a composition comprising delta-tocotrienol quinone and, optionally, a pharmaceutically acceptable carrier, diluent or excipient.

The present invention further relates to a composition comprising an effective amount of one or more compounds of Formula I or mixtures thereof and, optionally, a pharmaceutically acceptable carrier, diluent or excipient, for preventing, reducing, ameliorating or treating vascular disease complications of diabetes, including retinopathy and nephropathy, in a subject with abnormal glucose tolerance and metabolism in need of such treatment, where the one or more compounds of Formula I have the following structure:

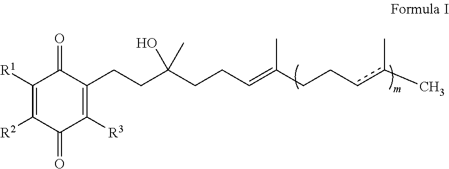

wherein,
each bond indicated by a dashed line can independently be double or single;
R¹, R² and R³ are independently of each other hydrogen, (C₁-C₆)alkyl or (C₁-C₆)alkoxy; and
m is an integer of 0 to 12 inclusive;
or any stereoisomer, mixture of stereoisomers, prodrug, metabolite, salt, crystalline form, non-crystalline form, hydrate or solvate thereof.

In some embodiments, the composition for preventing, reducing, ameliorating or treating vascular disease complications of diabetes, including retinopathy and nephropathy, is administered via oral, gastrointestinal, rectal, parenteral, local, inhalant or enteral route. In other embodiments, the composition for preventing, reducing, ameliorating or treating vascular disease complications of diabetes, including retinopathy and nephropathy, is an oral composition.

In some embodiments, the invention relates to a composition for preventing, reducing, ameliorating or treating vascular disease complications of diabetes, including retinopathy and nephropathy, comprising an effective amount of one or more compounds of Formula I, wherein R¹ and R² are independently of each other (C₁-C₆)alkoxy and R³ is (C₁-C₆)alkyl. In some embodiments, the invention relates to a composition for preventing, reducing, ameliorating or treating vascular disease complications of diabetes, including retinopathy and nephropathy, comprising an effective amount of one or more compounds of Formula I, wherein R¹ and R² are independently of each other (C₁-C₆)alkoxy and R³ is hydrogen. In other embodiments, the invention relates to a composition for preventing, reducing, ameliorating or treating vascular disease complications of diabetes, including retinopathy and nephropathy, comprising an effective amount of one or more compounds of Formula I, wherein R¹, R² and R³ are independently of each other (C₁-C₆)alkyl or hydrogen. In other embodiments, the invention relates to a composition comprising an effective amount of one or more compounds of Formula I, wherein R¹, R² and R³ are independently of each other (C₁-C₆)alkyl.

In some embodiments, the invention relates to a composition for preventing, reducing, ameliorating or treating vascular disease complications of diabetes, including retinopathy and nephropathy, comprising an effective amount of one or more compounds of Formula I, wherein R¹ and R² are independently of each other (C₁-C₄)alkoxy and R³ is (C₁-C₄)alkyl. In some embodiments, the invention relates to a composition for preventing, reducing, ameliorating or treating vascular disease complications of diabetes, including retinopathy and nephropathy, comprising an effective amount of one or more compounds of Formula I, wherein R¹ and R² are independently of each other (C₁-C₄)alkoxy and R³ is hydrogen. In other embodiments, the invention relates to a composition for preventing, reducing, ameliorating or treating vascular disease complications of diabetes, including retinopathy and nephropathy, comprising an effective amount of one or more compounds of Formula I, wherein R¹, R² and R³ are independently of each other (C₁-C₄)alkyl or hydrogen. In other embodiments, the invention relates to a composition for preventing, reducing, ameliorating or treating vascular disease complications of diabetes, including retinopathy and nephropathy, comprising an effective amount of one or more compounds of Formula I, wherein R¹, R² and R³ are independently of each other (C₁-C₄)alkyl.

In some embodiments, the invention relates to a composition for preventing, reducing, ameliorating or treating vascular disease complications of diabetes, including retinopathy and nephropathy, comprising an effective amount of one or more compounds of Formula I, wherein R¹ and R² are methoxy and R³ is methyl. In some embodiments, the invention relates to a composition for preventing, reducing, ameliorating or treating vascular disease complications of diabetes, including retinopathy and nephropathy, comprising an effective amount of one or more compounds of Formula I, wherein R¹ and R² are methoxy and R³ is hydrogen. In other embodiments, the invention relates to a composition for preventing, reducing, ameliorating or treating vascular disease complications of diabetes, including retinopathy and nephropathy, comprising an effective amount of one or more compounds of Formula I, wherein R¹, R² and R³ are independently of each other methyl or hydrogen. In other embodiments, the invention relates to a composition for preventing, reducing, ameliorating or treating vascular disease complications of diabetes, including retinopathy and nephropathy, comprising an effective amount of one or more compounds of Formula I, wherein R¹, R² and R³ are methyl.

In some embodiments, the invention relates to a composition for preventing, reducing, ameliorating or treating vascular disease complications of diabetes, including retinopathy and nephropathy, comprising an effective amount of one or more compounds of Formula I, wherein R¹ and R² are methoxy, R³ is methyl and m is an integer from 0-4 inclusive. In some embodiments, the invention relates to a composition for preventing, reducing, ameliorating or treating vascular disease complications of diabetes, including retinopathy and nephropathy, comprising an effective amount of one or more compounds of Formula I, wherein R¹ and R² are methoxy, R³ is hydrogen, and m is an integer from 0-4 inclusive. In other embodiments, the invention relates to a composition for preventing, reducing, ameliorating or treating vascular disease complications of diabetes, including retinopathy and nephropathy, comprising an effective amount of one or more compounds of Formula I, wherein R¹, R² and R³ are independently of each other methyl or hydrogen and m is an integer from 0-4 inclusive. In other embodiments, the invention relates to a composition for preventing, reducing, ameliorating or treating vascular disease complications of diabetes, including retinopathy and nephropathy, comprising an effective amount of one or more compounds of Formula I, wherein R¹, R² and R³ are methyl and m is an integer from 0-4 inclusive.

In some embodiments, the invention relates to a composition for preventing, reducing, ameliorating or treating vascular disease complications of diabetes, including retinopathy and nephropathy, comprising an effective amount of one or more compounds of Formula I, where the bond indicated by a dashed line is a double bond in every occurrence. In some embodiments, the invention relates to a composition for preventing, reducing, ameliorating or treating vascular disease complications of diabetes, including retinopathy and nephropathy, comprising an effective amount of one or more compounds of Formula I, where the bond indicated by a dashed line is a single bond in every occurrence.

In some embodiments, the invention relates to a composition for preventing, reducing, ameliorating or treating vascular disease complications of diabetes, including retinopathy and nephropathy, comprising an effective amount of a compound of Formula I wherein m is the integer zero. In some embodiments, the invention relates to a composition for preventing, reducing, ameliorating or treating vascular disease complications of diabetes, including retinopathy and nephropathy, comprising an effective amount of a compound of Formula I wherein m is the integer one. In some embodiments, the invention relates to a composition for preventing, reducing, ameliorating or treating vascular disease complications of diabetes, including retinopathy and nephropathy, comprising an effective amount of a compound of Formula I wherein m is the integer two. In some embodiments, the invention relates to a composition for preventing, reducing, ameliorating or treating vascular disease complications of diabetes, including retinopathy and nephropathy, comprising an effective amount of a compound of Formula I wherein m is the integer three. In some embodiments, the invention relates to a composition for preventing, reducing, ameliorating or treating vascular disease complications of diabetes, including retinopathy and nephropathy, comprising an effective amount of a compound of Formula I wherein m is the integer four. In some embodiments, the invention relates to a composition for preventing, reducing, ameliorating or treating vascular disease complications of diabetes, including retinopathy and nephropathy, comprising an effective amount of a compound of Formula I wherein m is the integer five. In some embodiments, the invention relates to a composition for preventing, reducing, ameliorating or treating vascular disease complications of diabetes, including retinopathy and nephropathy, comprising an effective amount of a compound of Formula I wherein m is the integer six. In some embodiments, the invention relates to a composition for preventing, reducing, ameliorating or treating vascular disease complications of diabetes, including retinopathy and nephropathy, comprising an effective amount of a compound of Formula I wherein m is the integer seven. In some embodiments, the invention relates to a composition for preventing, reducing, ameliorating or treating vascular disease complications of diabetes, including retinopathy and nephropathy, comprising an effective amount of a compound of Formula I wherein m is the integer eight. In some embodiments, the invention relates to a composition for preventing, reducing, ameliorating or treating vascular disease complications of diabetes, including retinopathy and nephropathy, comprising an effective amount of a compound of Formula I wherein m is the integer nine. In some embodiments, the invention relates to a composition for preventing, reducing, ameliorating or treating vascular disease complications of diabetes, including retinopathy and nephropathy, comprising an effective amount of a compound of Formula I wherein m is the integer ten. In some embodiments, the invention relates to a composition for preventing, reducing, ameliorating or treating vascular disease complications of diabetes, including retinopathy and nephropathy, comprising an effective amount of a compound of Formula I wherein m is the integer eleven. In some embodiments, the invention relates to a composition for preventing, reducing, ameliorating or treating vascular disease complications of diabetes, including retinopathy and nephropathy, comprising an effective amount of a compound of Formula I wherein m is the integer twelve. In any of the above embodiments, the bonds indicated by a dashed line in Formula I can be, independently of the other bonds, a double bond or a single bond. In any of the above embodiments, the bonds indicated by a dashed line in Formula I can be a double bond in every occurrence. In any of the above embodiments, the bonds indicated by a dashed line in Formula I can be a single bond in every occurrence.

In some embodiments, the invention relates to a composition for preventing, reducing, ameliorating or treating vascular disease complications of diabetes, including retinopathy and nephropathy, in a subject with abnormal glucose tolerance and metabolism, comprising an effective amount of a compound of Formula I, wherein the compound is selected from alpha-tocotrienol quinone, beta-tocotrienol quinone, gamma-tocotrienol quinone or delta-tocotrienol quinone or any stereoisomer or mixture of stereoisomers thereof. In some embodiments, the invention relates to a composition for preventing, reducing, ameliorating or treating vascular disease complications of diabetes, including retinopathy and nephropathy, in a subject with abnormal glucose tolerance and metabolism, comprising a mixture of two or more tocotrienol quinones selected from alpha-tocotrienol quinone, beta-tocotrienol quinone, gamma-tocotrienol quinone and delta-tocotrienol quinone or any stereoisomer or mixture of stereoisomers thereof.

In some embodiments, the invention relates to a composition for preventing, reducing, ameliorating or treating vascular disease complications of diabetes, including retinopathy and nephropathy, in a subject with abnormal glucose tolerance and metabolism, comprising alpha-tocotrienol quinone and, optionally, a pharmaceutically acceptable carrier, diluent or excipient. In some embodiments, the invention relates to a composition for preventing, reducing, ameliorating or treating vascular disease complications of diabetes, including retinopathy and nephropathy, in a subject with abnormal glucose tolerance and metabolism, comprising beta-tocotrienol quinone and, optionally, a pharmaceutically acceptable carrier, diluent or excipient. In some embodiments, the invention relates to a composition for preventing, reducing, ameliorating or treating vascular disease complications of diabetes, including retinopathy and nephropathy, in a subject with abnormal glucose tolerance and metabolism, comprising gamma-tocotrienol quinone and, optionally, a pharmaceutically acceptable carrier, diluent or excipient. In some embodiments, the invention relates to a composition for preventing, reducing, ameliorating or treating vascular disease complications of diabetes, including retinopathy and nephropathy, in a subject with abnormal glucose tolerance and metabolism, comprising delta-tocotrienol quinone and, optionally, a pharmaceutically acceptable carrier, diluent or excipient.

The present invention further relates to a method for preventing, reducing, ameliorating or treating vascular disease complications of diabetes, including retinopathy and nephropathy, in a subject with abnormal glucose tolerance and metabolism in need of such treatment, which comprises administering an effective amount of one or more compounds of Formula I or mixtures thereof and, optionally, a pharmaceutically acceptable carrier, diluent or excipient, where the one or more compounds of Formula I have the following structure:

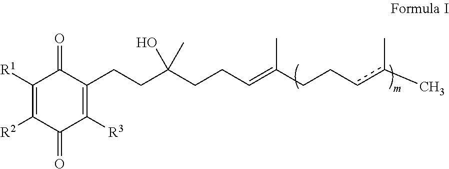

wherein,
each bond indicated by a dashed line can independently be double or single;
R¹, R² and R³ are independently of each other hydrogen, (C₁-C₆)alkyl or (C₁-C₆)alkoxy; and
m is an integer of 0 to 12 inclusive;
or any stereoisomer, mixture of stereoisomers, prodrug, metabolite, salt, crystalline form, non-crystalline form, hydrate or solvate thereof.

In some embodiments, the invention relates to a method for preventing, reducing, ameliorating or treating vascular disease complications of diabetes, including retinopathy and nephropathy, in a subject in need of treatment which comprises administering a composition via oral, gastrointestinal, rectal, parenteral, local, inhalant or enteral route. In other embodiments, the invention relates to a method for preventing, reducing, ameliorating or treating vascular disease complications of diabetes, including retinopathy and nephropathy, in a subject in need of such treatment which comprises administering an oral composition.

In some embodiments, the invention relates to a method for preventing, reducing, ameliorating or treating vascular disease complications of diabetes, including retinopathy and nephropathy, in a subject in need of such treatment which comprises administering a composition comprising an effective amount of one or more compounds of Formula I, wherein R¹ and R² are independently of each other (C₁-C₆)alkoxy and R³ is (C₁-C₆)alkyl. In some embodiments, the invention relates to a method for preventing, reducing, ameliorating or treating vascular disease complications of diabetes, including retinopathy and nephropathy, in a subject in need of such treatment which comprises administering a composition comprising an effective amount of one or more compounds of Formula I, wherein R¹ and R² are independently of each other (C₁-C₆)alkoxy and R³ is hydrogen. In other embodiments, the invention relates to a method for preventing, reducing, ameliorating or treating vascular disease complications of diabetes, including retinopathy and nephropathy, in a subject in need of such control which comprises administering a composition comprising an effective amount of one or more compounds of Formula I, wherein R¹, R² and R³ are independently of each other (C₁-C₆)alkyl or hydrogen. In other embodiments, the invention relates to a method for preventing, reducing, ameliorating or treating vascular disease complications of diabetes, including retinopathy and nephropathy, in a subject in need of such control which comprises administering a composition comprising an effective amount of one or more compounds of Formula I, wherein R¹, R² and R³ are independently of each other (C₁-C₆)alkyl.

In some embodiments, the invention relates to a method for preventing, reducing, ameliorating or treating vascular disease complications of diabetes, including retinopathy and nephropathy, in a subject in need of such treatment which comprises administering a composition comprising an effective amount of one or more compounds of Formula I, wherein R¹ and R² are independently of each other (C₁-C₄)alkoxy and R³ is (C₁-C₄)alkyl. In some embodiments, the invention relates to a method for preventing, reducing, ameliorating or treating vascular disease complications of diabetes, including retinopathy and nephropathy, in a subject in need of such treatment which comprises administering a composition comprising an effective amount of one or more compounds of Formula I, wherein R¹ and R² are independently of each other (C₁-C₄)alkoxy and R³ is hydrogen. In other embodiments, the invention relates to a method for preventing, reducing, ameliorating or treating vascular disease complications of diabetes, including retinopathy and nephropathy, in a subject in need of such control which comprises administering a composition comprising an effective amount of one or more compounds of Formula I, wherein R¹, R² and R³ are independently of each other (C₁-C₄)alkyl or hydrogen. In other embodiments, the invention relates to a method for preventing, reducing, ameliorating or treating vascular disease complications of diabetes, including retinopathy and nephropathy, in a subject in need of such control which comprises administering a composition comprising an effective amount of one or more compounds of Formula I, wherein R¹, R² and R³ are independently of each other (C₁-C₄)alkyl.

In some embodiments, the invention relates to a method for preventing, reducing, ameliorating or treating vascular disease complications of diabetes, including retinopathy and nephropathy, in a subject in need of such treatment which comprises administering a composition comprising an effective amount of one or more compounds of Formula I, wherein R¹ and R² are independently of each other methoxy and R³ is methyl. In some embodiments, the invention relates to a method for preventing, reducing, ameliorating or treating vascular disease complications of diabetes, including retinopathy and nephropathy, in a subject in need of such treatment which comprises administering a composition comprising an effective amount of one or more compounds of Formula I, wherein R¹ and R² are independently of each other methoxy and R³ is hydrogen. In other embodiments, the invention relates to a method for preventing, reducing, ameliorating or treating vascular disease complications of diabetes, including retinopathy and nephropathy, in a subject in need of such control which comprises administering a composition comprising an effective amount of one or more compounds of Formula I, wherein R¹, R² and R³ are methyl or hydrogen. In other embodiments, the invention relates to a method for preventing, reducing, ameliorating or treating vascular disease complications of diabetes, including retinopathy and nephropathy, in a subject in need of such control which comprises administering a composition comprising an effective amount of one or more compounds of Formula I, wherein R¹, R² and R³ are methyl.

In some embodiments, the invention relates to a method for preventing, reducing, ameliorating or treating vascular disease complications of diabetes, including retinopathy and nephropathy, in a subject in need of such treatment which comprises administering a composition comprising an effective amount of one or more compounds of Formula I, wherein R¹ and R² are methoxy, R³ is methyl and m is an integer from 0-4 inclusive. In some embodiments, the invention relates to a method for preventing, reducing, ameliorating or treating vascular disease complications of diabetes, including retinopathy and nephropathy, in a subject in need of such treatment which comprises administering a composition comprising an effective amount of one or more compounds of Formula I, wherein R¹ and R² are independently of each other methoxy, R³ is hydrogen and m is an integer from 0-4 inclusive. In other embodiments, the invention relates to a method for preventing, reducing, ameliorating or treating vascular disease complications of diabetes, including retinopathy and nephropathy, in a subject in need of such control which comprises administering a composition comprising an effective amount of one or more compounds of Formula I, wherein R¹, R² and R³ are methyl or hydrogen, and m is an integer from 0-4 inclusive. In other embodiments, the invention relates to a method for preventing, reducing, ameliorating or treating vascular disease complications of diabetes, including retinopathy and nephropathy, in a subject in need of such control which comprises administering a composition comprising an effective amount of one or more compounds of Formula I, wherein R¹, R² and R³ are methyl and m is an integer from 0-4 inclusive.

In some embodiments, the invention relates to a method for preventing, reducing, ameliorating or treating vascular disease complications of diabetes, including retinopathy and nephropathy, comprising administering an effective amount of one or more compounds of Formula I, where the bond indicated by a dashed line is a double bond in every occurrence. In some embodiments, the invention relates to a method for preventing, reducing, ameliorating or treating vascular disease complications of diabetes, including retinopathy and nephropathy, comprising administering an effective amount of one or more compounds of Formula I, where the bond indicated by a dashed line is a single bond in every occurrence.

In some embodiments, the invention relates to a method for preventing, reducing, ameliorating or treating vascular disease complications of diabetes, including retinopathy and nephropathy, in a subject in need of such treatment which comprises administering a composition comprising an effective amount of a compound of Formula I wherein m is the integer zero. In some embodiments, the invention relates to a method for preventing, reducing, ameliorating or treating vascular disease complications of diabetes, including retinopathy and nephropathy, in a subject in need of such treatment which comprises administering a composition comprising an effective amount of a compound of Formula I wherein m is the integer one. In some embodiments, the invention relates to a method for preventing, reducing, ameliorating or treating vascular disease complications of diabetes, including retinopathy and nephropathy, in a subject in need of such treatment which comprises administering a composition comprising an effective amount of a compound of Formula I wherein m is the integer two. In some embodiments, the invention relates to a method for preventing, reducing, ameliorating or treating vascular disease complications of diabetes, including retinopathy and nephropathy, in a subject in need of such treatment which comprises administering a composition comprising an effective amount of a compound of Formula I wherein m is the integer three. In some embodiments, the invention relates to a method for preventing, reducing, ameliorating or treating vascular disease complications of diabetes, including retinopathy and nephropathy, in a subject in need of such treatment which comprises administering a composition comprising an effective amount of a compound of Formula I wherein m is the integer four. In some embodiments, the invention relates to a method for preventing, reducing, ameliorating or treating vascular disease complications of diabetes, including retinopathy and nephropathy, in a subject in need of such treatment which comprises administering a composition comprising an effective amount of a compound of Formula I wherein m is the integer five. In some embodiments, the invention relates to a method for preventing, reducing, ameliorating or treating vascular disease complications of diabetes, including retinopathy and nephropathy, in a subject in need of such treatment which comprises administering a composition comprising an effective amount of a compound of Formula I wherein m is the integer six. In some embodiments, the invention relates to a method for preventing, reducing, ameliorating or treating vascular disease complications of diabetes, including retinopathy and nephropathy, in a subject in need of such treatment which comprises administering a composition comprising an effective amount of a compound of Formula I wherein m is the integer seven. In some embodiments, the invention relates to a method for preventing, reducing, ameliorating or treating vascular disease complications of diabetes, including retinopathy and nephropathy, in a subject in need of such treatment which comprises administering a composition comprising an effective amount of a compound of Formula I wherein m is the integer eight. In some embodiments, the invention relates to a method for preventing, reducing, ameliorating or treating vascular disease complications of diabetes, including retinopathy and nephropathy, in a subject in need of such treatment which comprises administering a composition comprising an effective amount of a compound of Formula I wherein m is the integer nine. In some embodiments, the invention relates to a method for preventing, reducing, ameliorating or treating vascular disease complications of diabetes, including retinopathy and nephropathy, in a subject in need of such treatment which comprises administering a composition comprising an effective amount of a compound of Formula I wherein m is the integer ten. In some embodiments, the invention relates to a method for preventing, reducing, ameliorating or treating vascular disease complications of diabetes, including retinopathy and nephropathy, in a subject in need of such treatment which comprises administering a composition comprising an effective amount of a compound of Formula I wherein m is the integer eleven. In some embodiments, the invention relates to a method for preventing, reducing, ameliorating or treating vascular disease complications of diabetes, including retinopathy and nephropathy, in a subject in need of such treatment which comprises administering a composition comprising an effective amount of a compound of Formula I wherein m is the integer twelve. In any of the above embodiments, the bonds indicated by a dashed line in Formula I can be, independently of the other bonds, a double bond or a single bond. In any of the above embodiments, the bonds indicated by a dashed line in Formula I can be a double bond in every occurrence. In any of the above embodiments, the bonds indicated by a dashed line in Formula I can be a single bond in every occurrence.

In some embodiments, the invention relates to a method for preventing, reducing, ameliorating or treating vascular disease complications of diabetes, including retinopathy and nephropathy, in a subject with abnormal glucose tolerance and metabolism in need of such treatment which comprises administering a composition comprising an effective amount of a compound of Formula I, wherein the compound is selected from alpha-tocotrienol quinone, beta- tocotrienol quinone, gamma-tocotrienol quinone or delta-tocotrienol quinone or any stereoisomer or mixture of stereoisomers thereof. In some embodiments, the invention relates to a method for preventing, reducing, ameliorating or treating vascular disease complications of diabetes, including retinopathy and nephropathy, in a subject with abnormal glucose tolerance and metabolism in need of such treatment, comprising administering a composition comprising an effective amount of a mixture of two or more tocotrienol quinones selected from alpha-tocotrienol quinone, beta-tocotrienol quinone, gamma-tocotrienol quinone and delta-tocotrienol quinone or any stereoisomer or mixture of stereoisomers thereof.

In some embodiments, the invention relates to a method for preventing, reducing, ameliorating or treating vascular disease complications of diabetes, including retinopathy and nephropathy, in a subject with abnormal glucose tolerance and metabolism in need of such treatment which comprises administering a composition comprising alpha-tocotrienol quinone and, optionally, a pharmaceutically acceptable carrier, diluent or excipient. In some embodiments, the invention relates to a method for preventing, reducing, ameliorating or treating vascular disease complications of diabetes, including retinopathy and nephropathy, in a subject with abnormal glucose tolerance and metabolism in need of such treatment which comprises administering a composition comprising beta-tocotrienol quinone and, optionally, a pharmaceutically acceptable carrier, diluent or excipient. In some embodiments, the invention relates to a method for preventing, reducing, ameliorating or treating vascular disease complications of diabetes, including retinopathy and nephropathy, in a subject with abnormal glucose tolerance and metabolism in need of such treatment which comprises administering a composition comprising gamma-tocotrienol quinone and, optionally, a pharmaceutically acceptable carrier, diluent or excipient. In some embodiments, the invention relates to a method for preventing, reducing, ameliorating or treating vascular disease complications of diabetes, including retinopathy and nephropathy, in a subject with abnormal glucose tolerance and metabolism in need of such treatment which comprises administering a composition comprising delta-tocotrienol quinone and, optionally, a pharmaceutically acceptable carrier, diluent or excipient.

The present invention further relates to a composition comprising an effective amount of one or more compounds of Formula I or mixtures thereof and, optionally, a pharmaceutically acceptable carrier, diluent or excipient, for preventing, reducing, ameliorating or treating inflammation in a subject with abnormal glucose tolerance and metabolism in need of such treatment, where the one or more compounds of Formula I have the following structure:

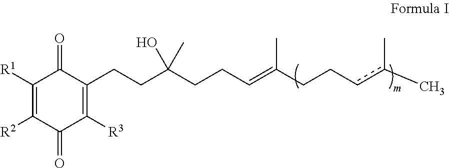

wherein,
each bond indicated by a dashed line can independently be double or single;
R¹, R² and R³ are independently of each other hydrogen, (C₁-C₆)alkyl or (C₁-C₆)alkoxy; and
m is an integer of 0 to 12 inclusive;
or any stereoisomer, mixture of stereoisomers, prodrug, metabolite, salt, crystalline form, non-crystalline form, hydrate or solvate thereof.

In some embodiments, the composition for preventing, reducing, ameliorating or treating inflammation is administered via oral, gastrointestinal, rectal, parenteral, local, inhalant or enteral route. In other embodiments, the composition for preventing, reducing, ameliorating or treating inflammation is an oral composition.

In some embodiments, the invention relates to a composition for preventing, reducing, ameliorating or treating inflammation comprising an effective amount of one or more compounds of Formula I, wherein R¹ and R² are independently of each other (C₁-C₆)alkoxy and R³ is (C₁-C₆)alkyl. In some embodiments, the invention relates to a composition for preventing, reducing, ameliorating or treating inflammation comprising an effective amount of one or more compounds of Formula I, wherein R¹ and R² are independently of each other (C₁-C₆)alkoxy and R³ is hydrogen. In other embodiments, the invention relates to a composition for preventing, reducing, ameliorating or treating inflammation comprising an effective amount of one or more compounds of Formula I, wherein R¹, R² and R³ are independently of each other (C₁-C₆)alkyl or hydrogen. In other embodiments, the invention relates to a composition comprising an effective amount of one or more compounds of Formula I, wherein R¹, R² and R³ are independently of each other (C₁-C₆)alkyl.

In some embodiments, the invention relates to a composition for preventing, reducing, ameliorating or treating inflammation comprising an effective amount of one or more compounds of Formula I, wherein R¹ and R² are independently of each other (C₁-C₄)alkoxy and R³ is (C₁-C₄)alkyl. In some embodiments, the invention relates to a composition for preventing, reducing, ameliorating or treating inflammation comprising an effective amount of one or more compounds of Formula I, wherein R¹ and R² are independently of each other (C₁-C₄)alkoxy and R³ is hydrogen. In other embodiments, the invention relates to a composition for preventing, reducing, ameliorating or treating inflammation comprising an effective amount of one or more compounds of Formula I, wherein R¹, R² and R³ are independently of each other (C₁-C₄)alkyl or hydrogen. In other embodiments, the invention relates to a composition for preventing, reducing, ameliorating or treating inflammation comprising an effective amount of one or more compounds of Formula I, wherein R¹, R² and R³ are independently of each other (C₁-C₄)alkyl.

In some embodiments, the invention relates to a composition for preventing, reducing, ameliorating or treating inflammation comprising an effective amount of one or more compounds of Formula I, wherein R¹ and R² are methoxy and R³ is methyl. In some embodiments, the invention relates to a composition for preventing, reducing, ameliorating or treating inflammation comprising an effective amount of one or more compounds of Formula I, wherein R¹ and R² are methoxy and R³ is hydrogen. In other embodiments, the invention relates to a composition for preventing, reducing, ameliorating or treating inflammation comprising an effective amount of one or more compounds of Formula I, wherein R¹, R² and R³ are independently of each other methyl or hydrogen. In other embodiments, the invention relates to a composition for preventing, reducing, ameliorating or treating inflammation comprising an effective amount of one or more compounds of Formula I, wherein R¹, R² and R³ are methyl.

In some embodiments, the invention relates to a composition for preventing, reducing, ameliorating or treating inflammation comprising an effective amount of one or more compounds of Formula I, wherein R¹ and R² are methoxy, R³ is methyl and m is an integer from 0-4 inclusive. In some embodiments, the invention relates to a composition for preventing, reducing, ameliorating or treating inflammation comprising an effective amount of one or more compounds of Formula I, wherein R¹ and R² are methoxy, R³ is hydrogen, and m is an integer from 0-4 inclusive. In other embodiments, the invention relates to a composition for preventing, reducing, ameliorating or treating inflammation comprising an effective amount of one or more compounds of Formula I, wherein R¹, R² and R³ are independently of each other methyl or hydrogen and m is an integer from 0-4 inclusive. In other embodiments, the invention relates to a composition for preventing, reducing, ameliorating or treating inflammation comprising an effective amount of one or more compounds of Formula I, wherein R¹, R² and R³ are methyl and m is an integer from 0-4 inclusive.

In some embodiments, the invention relates to a composition for preventing, reducing, ameliorating or treating inflammation comprising an effective amount of one or more compounds of Formula I, where the bond indicated by a dashed line is a double bond in every occurrence. In some embodiments, the invention relates to a composition for preventing, reducing, ameliorating or treating inflammation comprising an effective amount of one or more compounds of Formula I, where the bond indicated by a dashed line is a single bond in every occurrence.

In some embodiments, the invention relates to a composition for preventing, reducing, ameliorating or treating inflammation comprising an effective amount of a compound of Formula I wherein m is the integer zero. In some embodiments, the invention relates to a composition for preventing, reducing, ameliorating or treating inflammation comprising an effective amount of a compound of Formula I wherein m is the integer one. In some embodiments, the invention relates to a composition for preventing, reducing, ameliorating or treating inflammation comprising an effective amount of a compound of Formula I wherein m is the integer two. In some embodiments, the invention relates to a composition for preventing, reducing, ameliorating or treating inflammation comprising an effective amount of a compound of Formula I wherein m is the integer three. In some embodiments, the invention relates to a composition for preventing, reducing, ameliorating or treating inflammation comprising an effective amount of a compound of Formula I wherein m is the integer four. In some embodiments, the invention relates to a composition for preventing, reducing, ameliorating or treating inflammation comprising an effective amount of a compound of Formula I wherein m is the integer five. In some embodiments, the invention relates to a composition for preventing, reducing, ameliorating or treating inflammation comprising an effective amount of a compound of Formula I wherein m is the integer six. In some embodiments, the invention relates to a composition for preventing, reducing, ameliorating or treating inflammation comprising an effective amount of a compound of Formula I wherein m is the integer seven. In some embodiments, the invention relates to a composition for preventing, reducing, ameliorating or treating inflammation comprising an effective amount of a compound of Formula I wherein m is the integer eight. In some embodiments, the invention relates to a composition for preventing, reducing, ameliorating or treating inflammation comprising an effective amount of a compound of Formula I wherein m is the integer nine. In some embodiments, the invention relates to a composition for preventing, reducing, ameliorating or treating inflammation comprising an effective amount of a compound of Formula I wherein m is the integer ten. In some embodiments, the invention relates to a composition for preventing, reducing, ameliorating or treating inflammation comprising an effective amount of a compound of Formula I wherein m is the integer eleven. In some embodiments, the invention relates to a composition for preventing, reducing, ameliorating or treating inflammation comprising an effective amount of a compound of Formula I wherein m is the integer twelve. In any of the above embodiments, the bonds indicated by a dashed line in Formula I can be, independently of the other bonds, a double bond or a single bond. In any of the above embodiments, the bonds indicated by a dashed line in Formula I can be a double bond in every occurrence. In any of the above embodiments, the bonds indicated by a dashed line in Formula I can be a single bond in every occurrence.

In some embodiments, the invention relates to a composition for preventing, reducing, ameliorating or treating inflammation in a subject with abnormal glucose tolerance and metabolism, comprising an effective amount of a compound of Formula I, wherein the compound is selected from alpha-tocotrienol quinone, beta-tocotrienol quinone, gamma-tocotrienol quinone or delta-tocotrienol quinone or any stereoisomer or mixture of stereoisomers thereof. In some embodiments, the invention relates to a composition for preventing, reducing, ameliorating or treating inflammation in a subject with abnormal glucose tolerance and metabolism, comprising a mixture of two or more tocotrienol quinones selected from alpha-tocotrienol quinone, beta-tocotrienol quinone, gamma-tocotrienol quinone and delta-tocotrienol quinone or any stereoisomer or mixture of stereoisomers thereof.

In some embodiments, the invention relates to a composition for preventing, reducing, ameliorating or treating inflammation in a subject with abnormal glucose tolerance and metabolism, comprising alpha-tocotrienol quinone and, optionally, a pharmaceutically acceptable carrier, diluent or excipient. In some embodiments, the invention relates to a composition for preventing, reducing, ameliorating or treating inflammation in a subject with abnormal glucose tolerance and metabolism, comprising beta-tocotrienol quinone and, optionally, a pharmaceutically acceptable carrier, diluent or excipient. In some embodiments, the invention relates to a composition for preventing, reducing, ameliorating or treating inflammation in a subject with abnormal glucose tolerance and metabolism, comprising gamma-tocotrienol quinone and, optionally, a pharmaceutically acceptable carrier, diluent or excipient. In some embodiments, the invention relates to a composition for preventing, reducing, ameliorating or treating inflammation in a subject with abnormal glucose tolerance and metabolism, comprising delta-tocotrienol quinone and, optionally, a pharmaceutically acceptable carrier, diluent or excipient.

The present invention further relates to a method for preventing, reducing, ameliorating or treating inflammation in a subject with abnormal glucose tolerance and metabolism in need of such treatment, which comprises administering an effective amount of one or more compounds of Formula I or mixtures thereof and, optionally, a pharmaceutically acceptable carrier, diluent or excipient, where the one or more compounds of Formula I have the following structure:

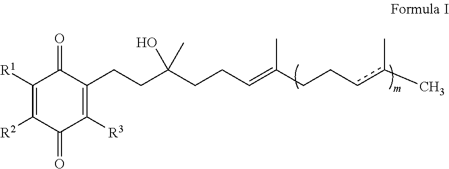

wherein,
each bond indicated by a dashed line can independently be double or single;
R¹, R² and R³ are independently of each other hydrogen, (C₁-C₆)alkyl or (C₁-C₆)alkoxy; and
m is an integer of 0 to 12 inclusive;
or any stereoisomer, mixture of stereoisomers, prodrug, metabolite, salt, crystalline form, non-crystalline form, hydrate or solvate thereof.

In some embodiments, the invention relates to a method for preventing, reducing, ameliorating or treating inflammation in a subject in need of treatment which comprises administering a composition via oral, gastrointestinal, rectal, parenteral, local, inhalant or enteral route. In other embodiments, the invention relates to a method for preventing, reducing, ameliorating or treating inflammation in a subject in need of such treatment which comprises administering an oral composition.

In some embodiments, the invention relates to a method for preventing, reducing, ameliorating or treating inflammation in a subject in need of such treatment which comprises administering a composition comprising an effective amount of one or more compounds of Formula I, wherein R¹ and R² are independently of each other (C₁-C₆)alkoxy and R³ is (C₁-C₆)alkyl. In some embodiments, the invention relates to a method for preventing, reducing, ameliorating or treating inflammation in a subject in need of such treatment which comprises administering a composition comprising an effective amount of one or more compounds of Formula I, wherein R¹ and R² are independently of each other (C₁-C₆)alkoxy and R³ is hydrogen. In other embodiments, the invention relates to a method for preventing, reducing, ameliorating or treating inflammation in a subject in need of such control which comprises administering a composition comprising an effective amount of one or more compounds of Formula I, wherein R¹, R² and R³ are independently of each other (C₁-C₆)alkyl or hydrogen. In other embodiments, the invention relates to a method for preventing, reducing, ameliorating or treating inflammation in a subject in need of such control which comprises administering a composition comprising an effective amount of one or more compounds of Formula I, wherein R¹, R² and R³ are independently of each other (C₁-C₆)alkyl.

In some embodiments, the invention relates to a method for preventing, reducing, ameliorating or treating inflammation in a subject in need of such treatment which comprises administering a composition comprising an effective amount of one or more compounds of Formula I, wherein R¹ and R² are independently of each other (C₁-C₄)alkoxy and R³ is (C₁-C₄)alkyl. In some embodiments, the invention relates to a method for preventing, reducing, ameliorating or treating inflammation in a subject in need of such treatment which comprises administering a composition comprising an effective amount of one or more compounds of Formula I, wherein R¹ and R² are independently of each other (C₁-C₄)alkoxy and R³ is hydrogen. In other embodiments, the invention relates to a method for preventing, reducing, ameliorating or treating inflammation in a subject in need of such control which comprises administering a composition comprising an effective amount of one or more compounds of Formula I, wherein R¹, R² and R³ are independently of each other (C₁-C₄)alkyl or hydrogen. In other embodiments, the invention relates to a method for preventing, reducing, ameliorating or treating inflammation in a subject in need of such control which comprises administering a composition comprising an effective amount of one or more compounds of Formula I, wherein R¹, R² and R³ are independently of each other (C₁-C₄)alkyl.

In some embodiments, the invention relates to a method for preventing, reducing, ameliorating or treating inflammation in a subject in need of such treatment which comprises administering a composition comprising an effective amount of one or more compounds of Formula I, wherein R¹ and R² are independently of each other methoxy and R³ is methyl. In some embodiments, the invention relates to a method for preventing, reducing, ameliorating or treating inflammation in a subject in need of such treatment which comprises administering a composition comprising an effective amount of one or more compounds of Formula I, wherein R¹ and R² are independently of each other methoxy and R³ is hydrogen. In other embodiments, the invention relates to a method for preventing, reducing, ameliorating or treating inflammation in a subject in need of such control which comprises administering a composition comprising an effective amount of one or more compounds of Formula I, wherein R¹, R² and R³ are methyl or hydrogen. In other embodiments, the invention relates to a method for preventing, reducing, ameliorating or treating inflammation in a subject in need of such control which comprises administering a composition comprising an effective amount of one or more compounds of Formula I, wherein R¹, R² and R³ are methyl.

In some embodiments, the invention relates to a method for preventing, reducing, ameliorating or treating inflammation in a subject in need of such treatment which comprises administering a composition comprising an effective amount of one or more compounds of Formula I, wherein R¹ and R² are methoxy, R³ is methyl and m is an integer from 0-4 inclusive. In some embodiments, the invention relates to a method for preventing, reducing, ameliorating or treating inflammation in a subject in need of such treatment which comprises administering a composition comprising an effective amount of one or more compounds of Formula I, wherein R¹ and R² are independently of each other methoxy, R³ is hydrogen and m is an integer from 0-4 inclusive. In other embodiments, the invention relates to a method for preventing, reducing, ameliorating or treating inflammation in a subject in need of such control which comprises administering a composition comprising an effective amount of one or more compounds of Formula I, wherein R¹, R² and R³ are methyl or hydrogen, and m is an integer from 0-4 inclusive. In other embodiments, the invention relates to a method for preventing, reducing, ameliorating or treating inflammation in a subject in need of such control which comprises administering a composition comprising an effective amount of one or more compounds of Formula I, wherein R¹, R² and R³ are methyl and m is an integer from 0-4 inclusive.

In some embodiments, the invention relates to a method for preventing, reducing, ameliorating or treating inflammation comprising administering an effective amount of one or more compounds of Formula I, where the bond indicated by a dashed line is a double bond in every occurrence. In some embodiments, the invention relates to a method for preventing, reducing, ameliorating or treating inflammation comprising administering an effective amount of one or more compounds of Formula I, where the bond indicated by a dashed line is a single bond in every occurrence.

In some embodiments, the invention relates to a method for preventing, reducing, ameliorating or treating inflammation in a subject in need of such treatment which comprises administering a composition comprising an effective amount of a compound of Formula I wherein m is the integer zero. In some embodiments, the invention relates to a method for preventing, reducing, ameliorating or treating inflammation in a subject in need of such treatment which comprises administering a composition comprising an effective amount of a compound of Formula I wherein m is the integer one. In some embodiments, the invention relates to a method for preventing, reducing, ameliorating or treating inflammation in a subject in need of such treatment which comprises administering a composition comprising an effective amount of a compound of Formula I wherein m is the integer two. In some embodiments, the invention relates to a method for preventing, reducing, ameliorating or treating inflammation in a subject in need of such treatment which comprises administering a composition comprising an effective amount of a compound of Formula I wherein m is the integer three. In some embodiments, the invention relates to a method for preventing, reducing, ameliorating or treating inflammation in a subject in need of such treatment which comprises administering a composition comprising an effective amount of a compound of Formula I wherein m is the integer four. In some embodiments, the invention relates to a method for preventing, reducing, ameliorating or treating inflammation in a subject in need of such treatment which comprises administering a composition comprising an effective amount of a compound of Formula I wherein m is the integer five. In some embodiments, the invention relates to a method for preventing, reducing, ameliorating or treating inflammation in a subject in need of such treatment which comprises administering a composition comprising an effective amount of a compound of Formula I wherein m is the integer six. In some embodiments, the invention relates to a method for preventing, reducing, ameliorating or treating inflammation in a subject in need of such treatment which comprises administering a composition comprising an effective amount of a compound of Formula I wherein m is the integer seven. In some embodiments, the invention relates to a method for preventing, reducing, ameliorating or treating inflammation in a subject in need of such treatment which comprises administering a composition comprising an effective amount of a compound of Formula I wherein m is the integer eight. In some embodiments, the invention relates to a method for preventing, reducing, ameliorating or treating inflammation in a subject in need of such treatment which comprises administering a composition comprising an effective amount of a compound of Formula I wherein m is the integer nine. In some embodiments, the invention relates to a method for preventing, reducing, ameliorating or treating inflammation in a subject in need of such treatment which comprises administering a composition comprising an effective amount of a compound of Formula I wherein m is the integer ten. In some embodiments, the invention relates to a method for preventing, reducing, ameliorating or treating inflammation in a subject in need of such treatment which comprises administering a composition comprising an effective amount of a compound of Formula I wherein m is the integer eleven. In some embodiments, the invention relates to a method for preventing, reducing, ameliorating or treating inflammation in a subject in need of such treatment which comprises administering a composition comprising an effective amount of a compound of Formula I wherein m is the integer twelve. In any of the above embodiments, the bonds indicated by a dashed line in Formula I can be, independently of the other bonds, a double bond or a single bond. In any of the above embodiments, the bonds indicated by a dashed line in Formula I can be a double bond in every occurrence. In any of the above embodiments, the bonds indicated by a dashed line in Formula I can be a single bond in every occurrence.

In some embodiments, the invention relates to a method for preventing, reducing, ameliorating or treating inflammation in a subject with abnormal glucose tolerance and metabolism in need of such treatment which comprises administering a composition comprising an effective amount of a compound of Formula I, wherein the compound is selected from alpha-tocotrienol quinone, beta-tocotrienol quinone, gamma-tocotrienol quinone or delta-tocotrienol quinone or any stereoisomer or mixture of stereoisomers thereof. In some embodiments, the invention relates to a method for preventing, reducing, ameliorating or treating inflammation in a subject with abnormal glucose tolerance and metabolism in need of such treatment, comprising administering a composition comprising an effective amount of a mixture of two or more tocotrienol quinones selected from alpha-tocotrienol quinone, beta-tocotrienol quinone, gamma-tocotrienol quinone and delta-tocotrienol quinone or any stereoisomer or mixture of stereoisomers thereof.

In some embodiments, the invention relates to a method for preventing, reducing, ameliorating or treating inflammation in a subject with abnormal glucose tolerance and metabolism in need of such treatment which comprises administering a composition comprising alpha-tocotrienol quinone and, optionally, a pharmaceutically acceptable carrier, diluent or excipient. In some embodiments, the invention relates to a method for preventing, reducing, ameliorating or treating inflammation in a subject with abnormal glucose tolerance and metabolism in need of such treatment which comprises administering a composition comprising beta-tocotrienol quinone and, optionally, a pharmaceutically acceptable carrier, diluent or excipient. In some embodiments, the invention relates to a method for preventing, reducing, ameliorating or treating inflammation in a subject with abnormal glucose tolerance and metabolism in need of such treatment which comprises administering a composition comprising gamma-tocotrienol quinone and, optionally, a pharmaceutically acceptable carrier, diluent or excipient. In some embodiments, the invention relates to a method for preventing, reducing, ameliorating or treating inflammation in a subject with abnormal glucose tolerance and metabolism in need of such treatment which comprises administering a composition comprising delta-tocotrienol quinone and, optionally, a pharmaceutically acceptable carrier, diluent or excipient.

In some of the aforementioned embodiments of the present invention, the methods may be used in treating conditions associated with abnormal glucose tolerance and metabolism, including insulin resistance, insulin resistant metabolic syndrome, impaired glucose tolerance, impaired fasting glucose, hypertension, hypercholesterolemia, obesity, and diabetes. In further embodiments of the present invention, the methods may be used in treating conditions associated with abnormal glucose tolerance, such as but not limited to impaired glucose tolerance (IGT) and impaired fasting glucose (IFG) where glucose values are above the conventional normal range and are often accompanied by a decrease of insulin sensitivity. In further embodiments of the present invention, the methods and compositions may be used in treating obesity and adiposity.

In some of the aforementioned embodiments of the present invention, the methods may be used for the treatment of a patient with abnormal blood glucose levels or insulin levels secondary to genetic disorders, such as genetic beta cells defects, maturity onset diabetes of the young (MODY), maternally inherited diabetes and deafness (MIDD), Werner's syndrome, ataxia telangiectasia, Donohue syndrome (leprechaunism), Mendenhall syndrome, Klinefelter's syndrome, Turner's syndrome and Down's syndrome.

In other of the aforementioned embodiments of the present invention, the subject in need of such treatment is suffering from gestational diabetes.

In some embodiments of the present invention, the patient is administered a daily dose of a composition comprising a tocotrienol quinone in a range of 0.1-100 mg per kg body weight of said subject. In yet other embodiments, a daily dose of composition comprises a tocotrienol quinone in a range of 0.1-50 mg per kg body weight of said subject. In further embodiments, a daily dose of composition comprises a tocotrienol quinone in a range of 0.5-25 mg per kg body weight of said subject.

In some embodiments of the present invention, the patient is administered a daily dose of a composition comprising an alpha-tocotrienol quinone in a range of 0.1-100 mg per kg body weight of said subject. In yet other embodiments, a daily dose of composition comprises an alpha-tocotrienol quinone in a range of 0.1-50 mg per kg body weight of said subject. In further embodiments, a daily dose of composition comprises an alpha-tocotrienol quinone in a range of 0.5-25 mg per kg body weight of said subject.

In some embodiments of the present invention, the patient is administered a daily dose of a composition comprising a beta-tocotrienol quinone in a range of 0.1-100 mg per kg body weight of said subject. In yet other embodiments, a daily dose of composition comprises a beta-tocotrienol quinone in a range of 0.1-50 mg per kg body weight of said subject. In further embodiments, a daily dose of composition comprises a beta-tocotrienol quinone in a range of 0.5-25 mg per kg body weight of said subject.

In some embodiments of the present invention, the patient is administered a daily dose of a composition comprising a gamma-tocotrienol quinone in a range of 0.1-100 mg per kg body weight of said subject. In yet other embodiments, a daily dose of composition comprises a gamma-tocotrienol quinone in a range of 1-50 mg per kg body weight of said subject. In further embodiments, a daily dose of composition comprises a gamma-tocotrienol quinone in a range of 0.5-25 mg per kg body weight of said subject.

In some embodiments of the present invention, the patient is administered a daily dose of a composition comprising a delta-tocotrienol quinone in a range of 0.1-100 mg per kg body weight of said subject. In yet other embodiments, a daily dose of composition comprises a delta-tocotrienol quinone in a range of 0.1-50 mg per kg body weight of said subject. In further embodiments, a daily dose of composition comprises a delta-tocotrienol quinone in a range of 0.5-25 mg per kg body weight of said subject.

The present invention also encompasses novel compositions and methods for making such compositions. In other embodiments, a composition is a pharmaceutical composition. In other embodiments, the pharmaceutical composition comprises a pharmaceutically acceptable carrier, diluent, or excipient. In other embodiments, a composition is a medical food. In some embodiments, the administering is via an enteral route. In other embodiments, the administering is via an oral route. In yet further embodiments, the administering is via a parenteral route.

In other aspects, the present invention provides tocotrienol quinone compositions comprising a composition enriched with alpha-tocotrienol quinone in an amount effective to reduce insulin resistance or increase insulin sensitivity related to an abnormal glucose tolerance and metabolism in a subject in need of such treatment.

In other aspects, the present invention provides tocotrienol quinone compositions comprising a composition enriched with beta-tocotrienol quinone in an amount effective to reduce insulin resistance or increase insulin sensitivity related to an abnormal glucose tolerance and metabolism in a subject in need of such treatment.

In other aspects, the present invention provides tocotrienol quinone compositions comprising a composition enriched with gamma-tocotrienol quinone in an amount effective to reduce insulin resistance or increase insulin sensitivity related to an abnormal glucose tolerance and metabolism in a subject in need of such treatment.

In other aspects, the present invention provides tocotrienol quinone compositions comprising a composition enriched with delta-tocotrienol quinone in an amount effective to reduce insulin resistance or increase insulin sensitivity related to an abnormal glucose tolerance and metabolism in a subject in need of such treatment.

In some of the aforementioned embodiments, the formulation comprises one or more of the following compounds:
alpha-tocotrienol quinone;
beta-tocotrienol quinone;
gamma-tocotrienol quinone;
delta-tocotrienol quinone;
2-[3-hydroxy-3,7,11,15,19,23,27,31,35,39-decamethyl-6,10,14,18,22,26,30,34,38-tetracontanonaen-1-yl]-5,6-dimethoxy-3-methyl-2,5-cyclohexadiene-1,4-dione;
2-(3-hydroxy-3,7,11,15,19,23,27-heptamethyl-6,10,14,18,22,26-octacosahexaenyl)-5,6-dimethoxy-3-methyl-p-benzoquinone;
2-(3-hydroxy-3,7-dimethyloct-6-en-1-yl)-3,5,6-trimethylcyclohexa-2,5-diene-1,4-dione;
5-(3-hydroxy-3,7,11-trimethyldodeca-6,10-dien-l-yl)-2,3-dimethylcyclohexa-2,5-diene-1,4-dione;
2-(3-hydroxy-3,7-dimethyloct-6-en-1-yl)-3,5,6-trimethylcyclohexa-2,5-diene-1,4-dione;
2-(3-hydroxy-3,7-dimethyloct-6-en-1-yl)-5,6-dimethoxy-3-methylcyclohexa-2,5-diene-1,4-dione;
2-(3-hydroxy-3,7,11-trimethyldodeca-6,10-dien-1-yl)-3,5,6-trimethylcyclohexa-2,5-diene-1,4-dione;
2-(3-hydroxy-3,7,11-trimethyldodec-6-en-1-yl)-3,5,6-trimethylcyclohexa-2,5-diene-1,4-dione;
2-(3-hydroxy-3,7,11-trimethyldodeca-6,10-dien-1-yl)-5,6-dimethoxy-3-methylcyclohexa-2,5-diene-1,4-dione;
2-(3-hydroxy-3,7,11-trimethyldodec-6-en-1-yl)-5,6-dimethoxy-3-methylcyclohexa-2,5-diene-1,4-dione;
2-(3-hydroxy-3,7,11,15-tetramethylhexadeca-6,10,14-trien-1-yl)-5,6-dimethoxy-3-methylcyclohexa-2,5-diene-1,4-dione;
2-(3-hydroxy-3,7,11,15-tetramethylhexadec-6-en-1-yl)-5,6-dimethoxy-3-methylcyclohexa-2,5-diene-1,4-dione;
5-(3-hydroxy-3,7,11,15-tetramethylhexadeca-6,10,14-trien-1-yl)-2,3-dimethoxycyclohexa-2,5-diene-1,4-dione;
2,3-diethyl-5-(3-hydroxy-3,7,11,15-tetramethylhexadeca-6,10,14-trien-1-yl)-6-methylcyclohexa-2,5-diene-1,4-dione;
2-(3-hydroxy-3,7,11,15-tetramethylhexadeca-6,10,14-trien-1-yl)-5,6-diisopropyl-3-methylcyclohexa-2,5-diene-1,4-dione;
5-(3-hydroxy-3,7,11,15,19,23-hexamethyltetracosa-6,10,14,18,22-pentaen-1-yl)-2,3-dimethoxycyclohexa-2,5-diene-1,4-dione;
2-(3-hydroxy-3,7,11,15,19,23-hexamethyltetracosa-6,10,14,18,22-pentaen-1-yl)-5,6-dimethoxy-3-methylcyclohexa-2,5-diene-1,4-dione;
2-(3-hydroxy-3,7,11,15,19,23-hexamethyltetracos-6-en-1-yl)-5,6-dimethoxy-3-methylcyclohexa-2,5-diene-1,4-dione;
2-(3-hydroxy-3,7,11,15,19,23-hexamethyltetracosa-6,10,14,18,22-pentaen-1-yl)-3,5,6-trimethylcyclohexa-2,5-diene-1,4-dione;
5-(3-hydroxy-3,7,11,15,19,23-hexamethyltetracosa-6,10,14,18,22-pentaen-1-yl)-2,3-dimethylcyclohexa-2,5-diene-1,4-dione;
2-(3-hydroxy-3,7,11,15,19,23,27,31,35-nonamethylhexatriaconta-6,10,14,18,22,26,30,34-octaen-1-yl)-3,5,6-trimethylcyclohexa-2,5-diene-1,4-dione;
2-(3-hydroxy-3,7,11,15,19,23,27,31,35-nonamethylhexatriaconta-6,10,14,18,22,26,30,34-octaen-1-yl)-5,6-dimethoxy-3-methylcyclohexa-2,5-diene-1,4-dione;
5-(3-hydroxy-3,7,11,15,19,23,27,31,35-nonamethylhexatriaconta-6,10,14,18,22,26,30,34-octaen-1-yl)-2,3-dimethoxycyclohexa-2,5-diene-1,4-dione;
2-(3-hydroxy-3,7,11,15,19,23,27,31,35-nonamethylhexatriaconta-6,10-dien-1-yl)-5,6-dimethoxy-3-methylcyclohexa-2,5-diene-1,4-dione; and
2-(3-hydroxy-3,7,11,15,19,23,27,31,35-nonamethylhexatriaconta-6,10-dien-1-yl)-3,5,6-trimethylcyclohexa-2,5-diene-1,4-dione;
   or any stereoisomer, mixture of stereoisomers, prodrug, metabolite, salt, crystalline form, non-crystalline form, hydrate or solvate thereof. In further embodiments, the compounds can be combined with a pharmaceutically acceptable carrier, diluent, or excipient.

For all of the compounds and methods described above, the quinone form can also be used in its reduced (hydroquinone, 1,4-benzenediol) form when desired. Likewise, the hydroquinone form can also be used in its oxidized (quinone) form when desired.

In some aspects, the present invention provides tocotrienol hydroquinone compositions comprising a composition of alpha-tocotrienol hydroquinone in an amount effective to reduce insulin resistance or increase insulin sensitivity related to an abnormal glucose tolerance and metabolism in a subject in need of such treatment.

In other aspects, the present invention provides tocotrienol hydroquinone compositions comprising a composition of beta-tocotrienol hydroquinone in an amount effective to reduce insulin resistance or increase insulin sensitivity related to an abnormal glucose tolerance and metabolism in a subject in need of such treatment.

In other aspects, the present invention provides tocotrienol hydroquinone compositions comprising a composition of gamma-tocotrienol hydroquinone in an amount effective to reduce insulin resistance or increase insulin sensitivity related to an abnormal glucose tolerance and metabolism in a subject in need of such treatment.

In other aspects, the present invention provides tocotrienol hydroquinone compositions comprising a composition of delta-tocotrienol hydroquinone in an amount effective to reduce insulin resistance or increase insulin sensitivity related to an abnormal glucose tolerance and metabolism in a subject in need of such treatment.

For all of the compounds and methods described herein, the invention also encompasses the use in treatment of the compounds and methods disclosed. The invention also encompasses the use of the compounds described herein for preparation of a medicament for use in preventing or treating a condition requiring increasing insulin sensitivity or reducing insulin resistance in a subject with abnormal glucose tolerance. The invention also encompasses the use of the compounds described herein for preparation of a medicament for use in preventing, reducing, ameliorating or treating diabetes, or in stabilizing the level of blood sugar, stopping the increase in level of blood sugar, reversing the increase in the level of blood sugar, or reducing the level of blood sugar in a subject with abnormal glucose tolerance and metabolism in need of such treatment.

The present invention comprises multiple aspects, features and embodiments, where such multiple aspects, features and embodiments, can be combined and permuted in any desired manner. These and other aspects, features and embodiments of the present invention will become evident upon reference to the remainder of this application, including the following detailed description. In addition, various references are set forth herein that describe in more detail certain compositions, and/or methods; all such references are incorporated herein by reference in their entirety.

### DETAILED DESCRIPTION OF THE INVENTION

### Modes for Carrying Out the Invention

The present invention generally relates to methods and compositions that can be used to treat conditions related to abnormal glucose tolerance and metabolism. The present invention provides compositions and methods for improving blood glucose control or for treating a condition requiring increasing insulin sensitivity or reducing insulin resistance. The present invention provides compositions and methods for reducing symptoms and/or conditions associated with abnormal levels of insulin, such as, for example, insulin resistance, insulin resistant metabolic syndrome, impaired glucose tolerance, impaired fasting glucose, hypertension, hypercholesterolemia, obesity, and diabetes.

The present invention provides tocotrienol quinone compositions comprising at least 50% alpha-tocotrienol quinone, at least 55% alpha-tocotrienol quinone, at least 60% alpha-tocotrienol quinone, at least 65% alpha-tocotrienol quinone, at least 70% alpha-tocotrienol quinone, at least 75% alpha-tocotrienol quinone, at least 80% alpha-tocotrienol quinone, at least 85% alpha-tocotrienol quinone, at least 90% alpha-tocotrienol quinone, at least 95% alpha-tocotrienol quinone and at least 98% alpha-tocotrienol quinone. In some embodiments, tocotrienol quinone compositions comprise alpha-tocotrienol quinone as the sole active ingredient. In preferred embodiments, an active ingredient is able to reduce symptoms and conditions associated with abnormal levels of insulin at least about 30%, at least about 40%, at least about 50%, at least about 60%, at least about 70%, at least about 80%, at least about 85%, and even more preferably at least about 90%, in experimental models such as those described herein

In additional preferred embodiments, the tocotrienol quinone composition comprises alpha-tocotrienol quinone in an amount effective to reduce abnormal levels of insulin in the blood and/or urine and may further comprise additional active ingredients. In some embodiments, the compositions comprising the tocotrienol quinone and additional ingredient(s) provide a synergistic effect. Tocotrienol quinone and an additional ingredient are considered to be synergistic when their combined effect is greater than additive of the individual effects. In other embodiments, the compositions comprise alpha-tocotrienol quinone and an additional ingredient providing a synergistic effect.

The monitoring of the effect of a treatment can be followed by the administration of the fasting plasma glucose test (FGT) or by the standard oral glucose tolerance test (OGTT). The fasting plasma glucose test consists of a simple, noninvasive blood test, where the results are interpreted by looking at glucose levels in the blood. In the standard oral glucose tolerance test 75 g of glucose are given to the fasted individual; the blood glucose levels and optionally insulin levels are measured every 30 minutes, usually for 2 or 3 hours.

The monitoring of the level of insulin sensitivity can also be measured for example with the insulin tolerance test (ITT), the frequently sampled intravenous glucose tolerance test (FSIGTT) and the euglycemic clamp test. The insulin tolerance test (ITT) is a medical diagnostic procedure during which insulin is injected into a patient's vein to assess pituitary function, adrenal function, and sometimes for other purposes. An ITT is usually ordered and interpreted by endocrinologists. The euglycemic clamp test involves infusing glucose at a variable rate in order to obtain a constant plasma glucose concentration. When insulin is also infused, the glucose infusion rate reflects insulin mediated glucose uptake.

### Definitions

As used in the present specification, the following words and phrases are generally intended to have the meanings as set forth below, except to the extent that the context in which they are used indicates otherwise.

The term “alkyl” refers to saturated aliphatic groups including straight-chain, branched-chain, cyclic groups, and combinations thereof, having the number of carbon atoms specified, or if no number is specified, having up to 12 carbon atoms. One subset of alkyl groups is (C₁-C₆)alkyl which include groups such as methyl, ethyl, n-propyl, isopropyl, butyl, n-butyl, isobutyl, sec-butyl, t-butyl, pentyl, n-pentyl, hexyl, cyclopropyl, cyclobutyl, cyclopentyl, cyclohexyl, and any other alkyl group containing between one and five carbon atoms, where the (C₁-C₆)alkyl groups can be attached via any valence on the (C₁-C₆)alkyl groups. Another subset of alkyl groups is (C₁-C₄)alkyl which include groups as methyl, ethyl, n-propyl, isopropyl, butyl, n-butyl, isobutyl, sec-butyl, t-butyl, cyclopropyl, and cyclobutyl.

The term “alkoxy” refers to the group —O-alkyl, wherein alkyl is as defined herein. One subset of alkoxy groups is (C₁-C₆)alkoxy which include groups such as methoxy, ethoxy, n-propoxy, iso-propoxy, cyclo-propoxy, n-butoxy, tert-butoxy, sec-butoxy, cyclo-butoxy, n-pentoxy, cyclopentoxy, n-hexoxy, cyclohexoxy, and 1,2-dimethylbutoxy. Another subset of alkoxy groups is (C₁-C₄)alkoxy which include groups such as methoxy, ethoxy, n-propoxy, iso-propoxy, cyclo-propoxy, n-butoxy, tert-butoxy, sec-butoxy, and cyclo-butoxy.

A “pharmaceutical composition” is a composition that is suitable for administration to a subject. A pharmaceutical composition may require a physician's prescription for administration. A pharmaceutical composition will comprise a compound of Formula I (or multiple compounds of Formula I) such as a tocotrienol quinone to be administered in a range of about 0.1 to about 100 mg per kg body weight of said mammalian subject per day. In some embodiments, a pharmaceutical composition further comprises a pharmaceutically acceptable carrier, diluent, or excipient.

A “medical food” is a product prescribed by a physician that is intended for the specific dietary management of a disorder or health condition for which distinctive nutritional requirements exist, and may include compositions fed through a feeding tube (referred to as enteral administration or gavage administration).

A “dietary supplement” shall mean a product that is intended to supplement the human diet and may be provided in the form of a pill, capsule, tablet, or like formulation. By way of example, but not limitation, a dietary supplement may include one or more of the following dietary ingredients: vitamins, minerals, herbs, botanicals, amino acids, and dietary substances intended to supplement the diet by increasing total dietary intake, or a concentrate, metabolite, constituent, extract, or combinations of these ingredients, not intended as a conventional food or as the sole item of a meal or diet. Dietary supplements may also be incorporated into food stuffs, such as functional foods designed to promote control of glucose levels.

A “functional food” is an ordinary food that has one or more components or ingredients incorporated into it to give a specific medical of physiological benefit, other than a purely nutritional effect.

“Special nutrition food” means ingredients designed for particular diet related to conditions or to support treatment of nutritional deficiencies.

By “treatment of” or “treating” is meant any treatment of a disease or disorder including: inhibiting the disease, that is, arresting or suppressing the development of clinical symptoms; and/or relieving the disease, that is, causing the regression of clinical symptoms. By “prevention of” or “preventing” a disease is meant preventing or protecting against the disease or disorder, that is, causing, the clinical symptoms of the disease not to develop. The subject for treatment and/or prevention is preferably a mammal, more preferably a human.

By “tocotrienol quinone composition” is meant a composition comprising alpha-, beta-, gamma- , and/or delta-tocotrienol quinone and mixtures thereof. The term tocotrienol quinone also includes single stereoisomers and mixtures of stereoisomers. For all the tocotrienol quinone described herein, the quinone form can also be used in its reduced (hydroquinone, 1,4-benzenediol) form when desired. Likewise, the hydroquinone form can also be used in its oxidized (quinone) form when desired.

“Diabetes” is a metabolic disease that is defined by the presence of chronically elevated levels of blood glucose. Diabetes is caused by abnormal metabolism of glucose, protein and lipids, due to a deficiency or insufficiency of the actions of insulin. Typical signs of diabetes include an abnormal increase in the serum glucose level over the normal range of the glucose level and an excretion of glucose in the urine. Classic symptoms of diabetes in adults include polyuria, polydipsia, ketonuria, rapid weight loss, other acute manifestations of hyperglycemia, and elevated levels of plasma glucose.

Type 1 diabetes (also called insulin-dependent diabetes (IDDM), juvenile diabetes, brittle diabetes, or sugar diabetes) is accompanied by reduction of insulin producing cells, and Type 2 (also called non-insulin-dependent diabetes (NIDDM)) is caused by insulin sensitivity reduction or insulin secretion reduction. Symptoms of Type 1 diabetes include, but are not limited to, high levels of sugar in the blood when tested, high levels of sugar in the urine when tested, unusual thirst, frequent urination, extreme hunger but loss of body weight, blurred vision, nausea and vomiting, extreme weakness and tiredness, and irritability and mood changes. Complications associated with Type 1 diabetes include, but are not limited to, hypoglycemia (blood sugar drops too low, called insulin reaction), hyperglycemia (blood sugar is too high, indicating diabetes is not well controlled), and ketoacidosis (diabetic coma or loss of consciousness due to untreated or under-treated diabetes).

Type 2 diabetes is characterized by insulin resistance, i.e., a failure of the normal metabolic response of peripheral tissues to the action of insulin. Insulin resistance refers to a condition wherein the insulin level required to exhibit insulin activity at the same level as a healthy person is much higher than that of the healthy person. It is a condition wherein the activity of insulin or sensitivity for insulin is reduced. In clinical terms, insulin resistance is when elevated blood glucose levels persist in the presence of normal or elevated levels of insulin, or when normal blood glucose levels persist in the presence of elevated levels of insulin. The hyperglycemia associated with Type 2 diabetes can be reversed or ameliorated by diet or weight loss sufficient to restore the sensitivity of the peripheral tissues to insulin. Progression of Type 2 diabetes includes increasing concentrations of blood glucose, coupled with a relative decrease in the rate of glucose-induced insulin secretion. Unlike the pancreatic beta cells in Type 1 diabetics, the beta cells of Type 2 diabetics retain the ability to synthesize and secrete insulin. Type 2 diabetes mellitus is often accompanied by obesity.

Type 2 diabetes also includes a non-insulin dependent diabetes mellitus of the young people, MODY (maturity-onset type of the diabetes in the young), and a morbid hyperglycemia caused by continuous administration of a steroid drug such as glucocorticoid (a steroid diabetes), or a hyperglycemia of Cushing Syndrome or an acromegaly because they are diabetes under normal or high level of insulin conditions. Diabetes mellitus also includes other specific types of diabetes mellitus and gestational diabetes mellitus.

There are other types of diabetes sometimes referred as Type 3 and Type 4 diabetes. Type 3 diabetes typically refers to diabetes that is due to a genetic defect in beta cells, genetically related insulin resistance, diseases of the pancreas, hormonal defects, or that is induced by chemicals or drugs. Type 4 diabetes refers to gestational diabetes that occurs in 2% to 5% of pregnancies.

“Impaired fasting glucose” (IFG), also named “impaired fasting glycaemia” refers to a condition in which the fasting blood glucose is elevated above what is considered normal levels but is not high enough to be classified as diabetes mellitus. It is considered a pre-diabetic state, associated with insulin resistance and increased risk of cardiovascular pathology, although of lesser risk than impaired glucose tolerance (IGT). IFG sometimes progresses to type 2 diabetes mellitus. There is a 50% risk over 10 years of progressing to overt diabetes. A recent study cited the average time for progression as less than three years. IFG is also a risk factor for mortality. The World Health Organization (WHO) criterion for impaired fasting glucose is a fasting plasma glucose level from 6.1 mmol/l (110 mg/dL) to 6.9 mmol/l (125 mg/dL).

“Impaired glucose tolerance” (IGT) refers to a pre-diabetic state of dysglycemia that is associated with insulin resistance and increased risk of cardiovascular pathology. IGT may precede type 2 diabetes mellitus by many years. IGT is also a risk factor for mortality. The criterion for IGT is two-hour glucose levels of 140 to 199 mg per dL (7.8 to 11.0 mmol) on the 75-g oral glucose tolerance test. A patient is said to be under the condition of IGT when he has an intermediately raised glucose level after 2 hours, but less than would qualify for type 2 diabetes mellitus. The fasting glucose may be either normal or mildly elevated.

“Insulin Sensitivity” is a measurement of the tissue response to insulin. Typically, it refers to insulin's ability to cause tissues to absorb glucose from the blood. A loss of insulin sensitivity, also called insulin resistance, is a core part of the metabolic disorder that affects many people in industrial nations. Insulin sensitivity can be measured by the insulin sensitivity index that measures the ability of endogenous insulin to lower glucose in extracellular fluids by inhibiting glucose release from the liver and stimulating the peripheral consumption of glucose. It can also be measured by the glucose-clamp technique, which measures the effect of changes in insulin concentration on glucose clearance-glucose uptake rate divided by plasma glucose concentration per unit of body surface area.

“Insulin Resistance” (IR) is the condition in which normal amounts of insulin are inadequate to produce a normal insulin response from fat, muscle and liver cells. Insulin resistance is characterized by increased glucose concentration in the blood, increased insulin concentration in the blood, decreased ability to metabolize glucose in response to insulin or a combination of any of the above. Insulin resistance is recognized as a component of several disorders, including extreme insulin-resistance syndromes, rare inherited disorders, type 2 diabetes mellitus, obesity, stress, infection; uremia, acromegaly, glucocorticoid excess, and pregnancy. The loss of insulin production in insulin resistance and diabetes results in increased blood glucose or hyperglycemia, which in turn can contribute to nephropathy, neuropathy and retinopathy.

Insulin resistance is associated with abnormalities in glucose and lipid metabolism, obesity, kidney disease, high blood pressure and increased risk for cardiovascular disease.

“Patient” refers to a subject, generally a mammal, particularly a human, in need of treatment of a condition, disorder or disease, e.g., diabetes. The term also includes a subject that is at risk for diabetes or has a pre-diabetic condition.

The susceptibility of a subject may be diagnosed, tested or identified by screening the patient. The screening may occur in a variety of ways but typically the screening involves measuring glucose levels in a patient. Glucose levels are expressed in milligrams of glucose per deciliter of blood (mg/dl) or millimoles per liter (mmol/L).

“Glucose Tolerance Test” refers to a diagnostic test well-known in the art in which blood samples are taken after the administration of glucose to determine how quickly the glucose is cleared from the blood. The test is usually used to test for diabetes, insulin resistance, and sometimes reactive hypoglycemia or rarer disorders of carbohydrate metabolism. In the most commonly performed version of the test, an oral glucose tolerance test (OGTT), a standard dose of glucose is ingested by mouth and blood levels are checked two hours later.

Typically, a blood sample of about 5 milliliters is taken from a patient after a ten to twelve hour fast. The patient repeats the fast another day and provides another blood sample. A patient without diabetes would typically produce a result of between about 80 mg/dl and about 120 mg/dl (or about 4 mmol/L to about 7 mmol/L). If a patient has two fasting glucose levels of 126 mg/dl (or 7 mmol/L), then the patient is typically diagnosed with diabetes.

Similar to the fasting glucose test is the oral glucose tolerance test (OGTT). The patient fasts for ten to twelve hours, then a blood sample is taken. A glucose dose of about 75 milligrams (or 100 milligrams if testing for gestational diabetes) is administered to the patient and blood samples are taken every thirty minutes for the next two hours. If the patient has a glucose level that is equal to or greater than 200 mg/dl (or 11.1 mmol/L), then the patient is diagnosed with diabetes.

An alternative to the fasting method is the two-hour postprandial plasma glucose (2 hrPPG) test. This test measures the amount of glucose in blood plasma after a person eats a meal loaded with a specific amount of sugar, typically about 75 milligrams. After two hours, the patient's glucose levels are ascertained by evaluating a blood sample. If a patient has a level of about 200 mg/dl (or 11.1 mmol/L), then the patient is diabetic.

An alternative to measuring the glucose levels in the blood is measuring certain hemoglobin levels. While this test is not traditionally used for diagnosis, it is indicative of a patient's susceptibility to diabetes. Specifically, the level of hemoglobin Alc (or HbAlc) is measured. About 90% of hemoglobin is hemoglobin A and about 8% of hemoglobin A is made up of minor components referred to as Alc, Alb, Ala1, and Ala2. HbAlc is formed by a covalent reaction between glucose and hemoglobin A, and HbAlc levels depend on the blood glucose concentration. That is, the higher the glucose concentration in blood, the higher the level of HbAlc. This test has an advantage over the other tests in that the levels of HbAlc are not affected by short-term fluctuations in blood glucose. HbAlc can be easily measured by high performance liquid chromatography (HPLC) because it carries a different charge and also differs in size compared to other hemoglobin. The National Institutes of Health of the United States defines a normal level of HbAlc as less than 5.7%, a pre-diabetic level as 5.7%-6.4%, and a diabetic level as 6.5% or higher; see www.nlm.nih.gov/medlineplus/ency/article/003640.htm.

Even if no specific diagnostic test is administered, a patient is diabetic if the patient exhibits a random blood glucose of greater than 200 mg/dl (or 11.1 mmol/L) and exhibits known symptoms of diabetes, as described above.

“Pharmaceutically effective amount” or “therapeutically effective amount” refers to that amount of a compound of the present invention that is sufficient to effect treatment, as defined below, when administered to a subject in need of such treatment. The therapeutically effective amount will vary depending upon the subject and disease condition being treated, the weight and age of the subject, the severity of the disease condition, the particular compound chosen, the dosing regimen to be followed, timing of administration, the manner of administration and the like, all of which can readily be determined by one of ordinary skill in the art.

“Treatment” or “treating” means any treatment of a disease or disorder in a mammal, including: preventing or protecting against the disease or disorder, that is, causing the clinical symptoms not to develop; inhibiting the disease or disorder, that is, arresting or suppressing the development of clinical symptoms; and/or relieving the disease or disorder that is causing the regression of clinical symptoms.

It will be understood by those skilled in the art that in human medicine, it is not always possible to distinguish between “preventing” and “suppressing” since the ultimate inductive event or events may be unknown, latent, or the status of the patient is not ascertained until well after the occurrence of the event or events. Therefore, as used herein, the term “prophylaxis” is intended as an element of “treatment” to encompass both “preventing” and “suppressing” as defined herein.

“Protection,” as used herein, is meant to include “prophylaxis.”

It is also contemplated that the composition comprising a pharmaceutically effective amount of one or more compounds of Formula I may be co-administered with one or more additional diabetic medications. By “co-administered” is meant that the two (or more) agents are in clinical association with one another. Co-administration can include administering the agents together or sequentially. The co-administration may take place by the same delivery route or may be by separate delivery routes, for example, the composition comprising one or more compounds of Formula I may be administered peritoneally, while the other diabetic medication or medications may be administered orally. By “diabetic medication” is meant that a medication or pharmaceutical that prevents or treats diabetes or ameliorates the symptoms of diabetes. Diabetic medications, include, but are not limited to pioglitazone, glimepiride, rosiglitazone, glipizide, miglitol, repaglinide, acarbose, troglitazone, nateglinide, and combinations thereof.

### General Methods

The compositions of the present invention comprise tocotrienol quinones which can be produced synthetically from the respective tocotrienol by oxidation with suitable oxidizing agents, as for example ceric ammonium nitrate (CAN). Particularly, the compositions of the present invention comprise alpha-tocotrienol quinone (CAS Reg. No. 1401-66-7) produced by oxidation of essentially pure alpha-tocotrienol. A preferred process for the production of essentially pure alpha-tocotrienol has been described in co-owned US Patent Application Publication No. US 2010/0105930 titled “Process for the Production of Alpha-Tocotrienol and Derivatives.”

For all of the compounds and methods described herein, the quinone form can also be used in its reduced (hydroquinone, 1,4-benzenediol) form when desired. Likewise, the hydroquinone form can also be used in its oxidized (quinone) form when desired.

In one aspect, tocotrienol quinones are contemplated for use in treatment, including alpha-tocotrienol quinone, beta-tocotrienol quinone, gamma-tocotrienol quinone, and delta-tocotrienol quinone. In another aspect, alpha-tocotrienol quinone is contemplated for use in treatment. Structures of tocotrienol quinones are given in Table 1 below. The tocotrienol quinones with the naturally occurring tocotrienol configuration are used in one embodiment of the invention, but other stereoisomers and/or mixtures of stereoisomers in any ratio, such as racemic mixtures, can also be used in the invention.

Tocotrienol quinones can be used in their oxidized form, as shown in Table 1, or can be used in their reduced hydroquinone form, as shown in Table 2. The quinone (cyclohexadienedione) form and hydroquinone (benzenediol) form are readily interconverted with appropriate reagents. The quinone can be treated in a biphasic mixture of an ethereal solvent with a basic aqueous solution of Na₂S₂O₄ (Vogel, A. I. et al. Vogel's Textbook of Practical Organic Chemistry, 5th Edition, Prentice Hall: New York, 1996; Section 9.6.14 Quinones, “Reduction to the Hydroquinone”). Standard workup in the absence of oxygen yields the desired hydroquinone. The hydroquinone form can be oxidized to the quinone form with oxidizing agents such as ceric ammonium nitrate (CAN) or ferric chloride. The quinone and hydroquinone forms are also readily interconverted electrochemically, as is well known in the art. See, e.g., Section 33.4 of Streitweiser & Heathcock, Introduction to Organic Chemistry, New York: Macmillan, 1976.

TABLE 1
Tocotrienol quinones

| | |
|---|---|
| Alpha-tocotrienol quinone | 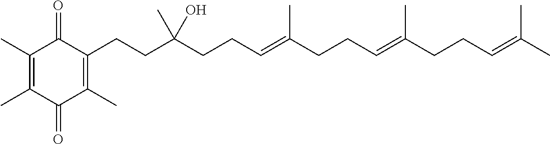 |
| Beta-tocotrienol quinone | 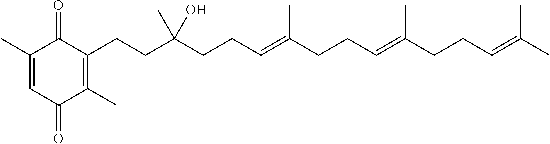 |
| Gamma-tocotrienol quinone | 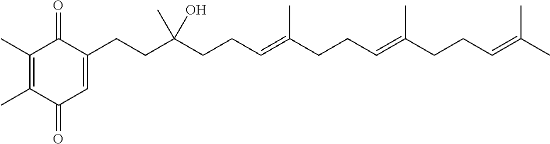 |
| Delta-tocotrienol quinone | 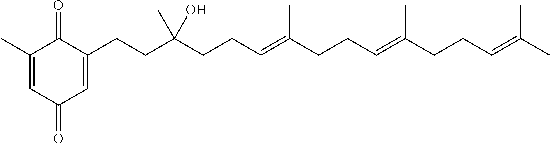 |

TABLE 2
Tocotrienol hydroquinones

| | |
|---|---|
| Alpha-tocotrienol hydroquinone | 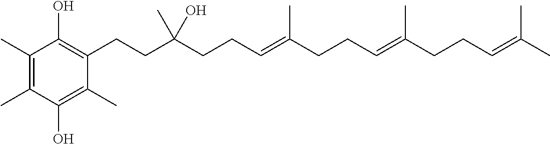 |
| Beta-tocotrienol hydroquinone | 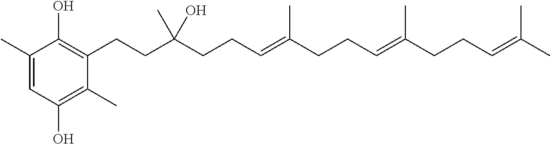 |
| Gamma-tocotrienol hydroquinone | 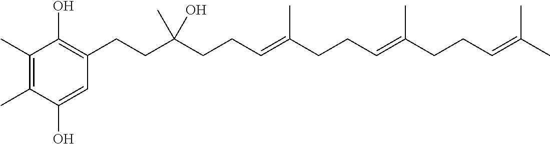 |
| Delta-tocotrienol hydroquinone | 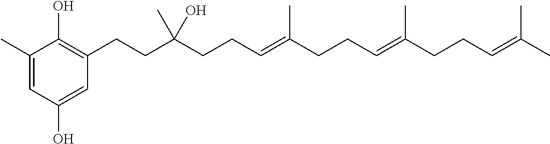 |

Illustrative examples provided herein are alpha-tocotrienol quinone-enriched compositions comprising alpha-tocotrienol quinone that may further comprise other tocotrienol quinones, e.g., beta-, gamma-, and delta-tocotrienol quinone, for use in the improvement of glucose control or in preventing or treating a condition requiring increasing insulin sensitivity or reducing insulin resistance in patients in need of such treatment.

These compounds are present in the compositions in amounts effective to ameliorate the injury and/or symptoms associated with insulin disorders. Alpha-tocotrienol quinone compositions comprise at least 50% alpha-tocotrienol quinone, at least 55% alpha-tocotrienol quinone, at least 60% alpha-tocotrienol quinone, at least 65% alpha-tocotrienol quinone, at least 70% alpha-tocotrienol quinone, at least 75% alpha-tocotrienol quinone, at least 80% alpha-tocotrienol quinone, at least 85% alpha-tocotrienol quinone, at least 90% alpha-tocotrienol quinone and at least 95% alpha-tocotrienol quinone.

In the present invention, a single dose of composition will comprise a tocotrienol quinone to be administered in a range of about 1 to about 100 mg per kg body weight of said mammalian subject. In additional embodiments, a composition will comprise a tocotrienol quinone to be administered at a lower limit of at least about 1, 10, 20, 30, 40, 50, 60, 70, 80, 90, or 100 mg per kg body weight of said mammalian subject. In additional embodiments, a pharmaceutical composition will comprise a tocotrienol quinone to be administered in a range of about 5 to about 100 mg per kg body weight of said mammalian subject.

### Testing

This section describes how compositions incorporating compositions of the present invention are selected using in vitro and in vivo animal models, and used as therapeutic interventions in diabetic indications.

In vivo evaluation of the role of compounds of Formula I, in particular of alpha-tocotrienol quinone in the reduction of serum glucose levels and insulin is performed in C57BLKs/J-m+/+Lepr db mice, a non-insulin dependent diabetic mellitus (NIDDM) model, as described in Examples.

### Formulation and Administration

The methods described herein use the compounds described herein formulated as pharmaceutical compositions by composition with additives such as pharmaceutically acceptable excipients, pharmaceutically acceptable carriers, and pharmaceutically acceptable vehicles. Suitable pharmaceutically acceptable excipients, carriers and vehicles include processing agents and drug delivery modifiers and enhancers, such as, for example, calcium phosphate, magnesium stearate, talc, monosaccharides, disaccharides, starch, gelatin, cellulose, methyl cellulose, sodium carboxymethyl cellulose, dextrose, hydroxypropyl-β-cyclodextrin, polyvinylpyrrolidinone, low melting waxes, ion exchange resins, and the like, as well as combinations of any two or more thereof. Other suitable pharmaceutically acceptable excipients are described in “Remington's Pharmaceutical Sciences,” Mack Pub. Co., New Jersey (1991), and “Remington: The Science and Practice of Pharmacy,” Lippincott Williams & Wilkins, Philadelphia, 20th edition (2003) and 21st edition (2005), incorporated herein by reference.

Pharmaceutical compositions containing the compounds of the invention may be in any form suitable for the intended method of administration, including, for example, a solution, a suspension, or an emulsion. Liquid carriers are typically used in preparing solutions, suspensions, and emulsions. Liquid carriers contemplated for use in the practice of the present invention include, for example, water, saline, pharmaceutically acceptable organic solvent(s), pharmaceutically acceptable oils or fats, and the like, as well as mixtures of two or more thereof. The liquid carrier may contain other suitable pharmaceutically acceptable additives such as solubilizers, emulsifiers, nutrients, buffers, preservatives, suspending agents, thickening agents, viscosity regulators, stabilizers, and the like. Suitable organic solvents include, for example, monohydric alcohols, such as ethanol, and polyhydric alcohols, such as glycols. Suitable oils include, for example, soybean oil, coconut oil, olive oil, safflower oil, cottonseed oil, and the like. For parenteral administration, the carrier can also be an oily ester such as ethyl oleate, isopropyl myristate, and the like. Compositions of the present invention may also be in the form of microparticles, microcapsules, liposomal encapsulates, and the like, as well as combinations of any two or more thereof.

Time-release or controlled release delivery systems may be used, such as a diffusion controlled matrix system or an erodible system, as described for example in: Lee, “Diffusion-Controlled Matrix Systems”, pp. 155-198 and Ron and Langer, “Erodible Systems”, pp. 199-224, in “Treatise on Controlled Drug Delivery”, A. Kydonieus Ed., Marcel Dekker, Inc., New York 1992. The matrix may be, for example, a biodegradable material that can degrade spontaneously in situ and in vivo for, example, by hydrolysis or enzymatic cleavage, e.g., by proteases. The delivery system may be, for example, a naturally occurring or synthetic polymer or copolymer, for example in the form of a hydrogel. Exemplary polymers with cleavable linkages include polyesters, polyorthoesters, polyanhydrides, polysaccharides, polyphosphoesters, polyamides, polyurethanes, polyimidocarbonates and polyphosphazenes.

The compounds of the invention may be administered enterally, orally, parenterally, sublingually, by inhalation (e.g. as mists or sprays), rectally, or topically in dosage unit formulations containing conventional nontoxic pharmaceutically acceptable carriers, adjuvants, and vehicles as desired. For example, suitable modes of administration include oral, subcutaneous, transdermal, transmucosal, iontophoretic, intravenous, intraarterial, intramuscular, intraperitoneal, intranasal (e.g. via nasal mucosa), subdural, rectal, gastrointestinal, and the like, and directly to a specific or affected organ or tissue. Topical administration may also involve the use of transdermal administration such as transdermal patches or iontophoresis devices. The term parenteral as used herein includes subcutaneous injections, intravenous, intramuscular, intrasternal injection, or infusion techniques. The compounds are mixed with pharmaceutically acceptable carriers, adjuvants, and vehicles appropriate for the desired route of administration. Oral administration is a preferred route of administration, and formulations suitable for oral administration are preferred formulations. The compounds described for use herein can be administered in solid form, in liquid form, in aerosol form, or in the form of tablets, pills, powder mixtures, capsules, granules, injectables, creams, solutions, suppositories, enemas, colonic irrigations, emulsions, dispersions, food premixes, and in other suitable forms. The compounds can also be administered in liposome formulations. Additional methods of administration are known in the art.

Injectable preparations, for example, sterile injectable aqueous or oleaginous suspensions, may be formulated according to the known art using suitable dispersing or wetting agents and suspending agents. The sterile injectable preparation may also be a sterile injectable solution or suspension in a nontoxic parenterally acceptable diluent or solvent, for example, as a solution in propylene glycol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid find use in the preparation of injectables.

Suppositories for rectal administration of the drug can be prepared by mixing the drug with a suitable nonirritating excipient such as cocoa butter and polyethylene glycols that are solid at room temperature but liquid at the rectal temperature and will therefore melt in the rectum and release the drug.

Solid dosage forms for oral administration may include capsules, tablets, pills, powders, and granules. In such solid dosage forms, the active compound may be admixed with at least one inert diluent such as sucrose, lactose, or starch. Such dosage forms may also comprise additional substances other than inert diluents, e.g., lubricating agents such as magnesium stearate. In the case of capsules, tablets, and pills, the dosage forms may also comprise buffering agents. Tablets and pills can additionally be prepared with enteric coatings.

Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, cyclodextrins, and sweetening, flavoring, and perfuming agents.

The compounds of the present invention can also be administered in the form of liposomes. As is known in the art, liposomes are generally derived from phospholipids or other lipid substances. Liposomes are formed by mono- or multilamellar hydrated liquid crystals that are dispersed in an aqueous medium. Any non-toxic, physiologically acceptable and metabolizable lipid capable of forming liposomes can be used. The present compositions in liposome form can contain, in addition to a compound of the present invention, stabilizers, preservatives, excipients, and the like. The preferred lipids are the phospholipids and phosphatidyl cholines (lecithins), both natural and synthetic. Methods to form liposomes are known in the art. See, for example, Prescott, Ed., Methods in Cell Biology, Volume XIV, Academic Press, New York, N.W., p. 33 et seq (1976).

The amount of the composition ingested, consumed or otherwise administered will depend on the desired final concentration. Typically, the amount of a single administration of the composition of the invention can be about 10 to about 1500 mg per day. Any of these doses can be further subdivided into separate administrations, and multiple dosages can be given to any individual patient.

The precise effective amount will vary from subject to subject and will depend upon the species, age, the subject's size and health, the nature and extent of the condition being treated, recommendations of the treating physician, and the therapeutics or combination of therapeutics selected for administration. The subject may be administered as many doses as is required to reduce and/or alleviate the signs, symptoms, or causes of the disorder in question, or bring about any other desired alteration of a biological system

The compounds of this invention may also be administered as compositions prepared as foods for humans or animals, including medical foods, functional food, special nutrition foods and dietary supplements.

Generally, depending on the intended mode of administration, the pharmaceutically acceptable composition will contain about 0.1% to 90%, for example about 0.5% to 50%, by weight of a compound or salt of compound of the present invention, the remainder being suitable pharmaceutical excipients, carriers, etc.

For administration, the formulations may conveniently be presented in unit dosage form and may be prepared by any methods well known in the art of pharmacy. Such methods include the step of bringing into association the active ingredients with the carrier which constitutes one or more accessory ingredients. In general, the formulations are prepared by uniformly and intimately bringing into association the active ingredients with liquid carriers or finely divided solid carriers or both, and then if necessary shaping the product. The invention also provides articles of manufacture and kits containing materials useful for treating or suppressing a condition related to high levels of blood sugar or a condition requiring increasing insulin sensitivity or reducing insulin resistance. The article of manufacture comprises a container with a label. Suitable containers include, for example, bottles, vials, and test tubes. The containers may be formed from a variety of materials such as glass or plastic. The container holds a composition having an active agent which is effective for treating or suppressing a condition related to high levels of blood sugar or a condition requiring increasing insulin sensitivity or reducing insulin resistance. The active agent in the composition is one or more of the compounds of the present invention. The label on the container indicates that the composition is used for treating or suppressing a condition related to high levels of blood sugar or a condition requiring increasing insulin sensitivity or reducing insulin resistance, for example diabetes, and may also indicate directions for either in vivo or in vitro use.

The invention also provides kits comprising any one or more of the compounds of the present invention. In some embodiments, the kit of the invention comprises the container described above. In other embodiments, the kit of the invention comprises the container described above and a second container comprising a buffer. It may further include other materials desirable from a commercial and user standpoint, including other buffers, diluents, filters, needles, syringes, and package inserts with instructions for performing any methods described herein.

In other aspects, the kits may be used for any of the methods described herein, including, for example, to treat an individual with a condition related to high levels of blood sugar, or to suppress a condition requiring increasing insulin sensitivity or reducing insulin resistance in an individual.

The above-mentioned compositions and methods of administration are meant to describe but not limit the methods and compositions of the present invention. The methods of producing various compositions and devices are within the ability of one skilled in the art and are not described in detail here.

### EXAMPLES

The following preparations and examples are given to enable those skilled in the art to more clearly understand and to practice the present invention. They should not be considered as limiting the scope of the invention, but merely as being illustrative and representative thereof.

### Example 1

### Therapeutic Study: Serum Glucose Levels in Diabetic Animals

### Animals

Non-insulin dependent diabetic mellitus (NIDDM) male mice (C57BLKS/J-m+/+Lepr db), weighing 45.+−0.5 g (10 weeks of age) and provided by Institute for Animal Reproduction (IAR, Japan), are used. These animals exhibited hyperinsulinemia, hyperglycemia and islet atrophy.

### Chemicals

Dimethylsulfoxide (DMSO, MERCK Germany),
ELISA Insulin assay kit (SPI bio, France),
Glucose (Merck, Germany),
Glucose-HA Assay kit (Wako, Japan),
Methylcellulose (Sigma, USA),
Sodium Chloride (Wako, Japan),
Tween 80 (Wako, Japan)

### Methods

Groups of 10 non-insulin dependent diabetic mellitus (NIDDM) male mice (C57BLKS/J-m+/+Lepr db), weighing 45 +/−0.5 g and 10 weeks old, are used. The animals are housed in Individually Ventilated Cages Racks (IVC Racks) throughout the experiment; all animals are allowed free access to sterilized Lab chow and sterilized distilled water. The test compounds, as well as vehicle (0.5% DMSO/2% Tween 80/0.9% NaCl), are each administered intraperitoneally twice daily (1st at 9:00 A.M. and 2nd at 16:00 P.M.) for a total of 14 consecutive days. Using blood samples collected from the orbital sinus, pre-treatment serum glucose and insulin levels are determined 24 hours prior to the first dosing on day 0. Post-treatment glucose and insulin levels are detected 4 hours after dosing on days 11 and 14. Also, serum glucose and insulin levels are obtained before and 15, 30 and 90 minutes after glucose loading (orally glucose tolerance test (OGTT) 2 g/kg), 4 hours following the dosing on day 11. Serum glucose and Insulin (on days 0, 11 and 14) levels are determined by enzymatic (Mutaratase-GOD) and ELISA (mouse insulin assay kit) methods, respectively. The percentage of post-treatment relative to pre-treatment group values obtained on days 11 and 14 are analyzed using One Way ANOVA followed by Dunnett's test for comparison between vehicle and treated groups. The differences are considered significant at p<0.05.

For effect on OGTT, AUC (area under the curve) for the serum glucose at times 0, 15, 30, and 90 minutes after OGTT is plotted for each test compound and its respective vehicle control. Body weight and food intake are measured daily through the course of the study.

Serum specimens on day 0 and day 14 are obtained by centrifuging blood samples at 3000 rpm, 4° C. for 10 minutes. Subsequently, the fat depots and liver are removed with a surgical excision and is then frozen at -80° C. until analysis.

What is claimed is:

1. A composition comprising one or more compounds of Formula I in an amount effective for improving blood glucose control in a subject with abnormal glucose tolerance and metabolism in need of such treatment, where the compounds of Formula I have the following structure: 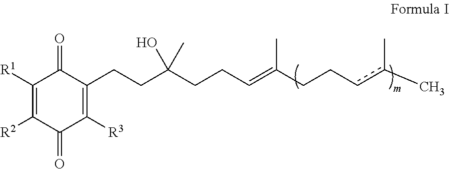
wherein,
each bond indicated by a dashed line can independently be double or single;
R¹, R², R³ are independently of each other hydrogen, (C₁-C₆)alkyl or (C₁-C₆)alkoxy; and
m is an integer of 0 to 12 inclusive;
or any stereoisomer, mixture of stereoisomers, prodrug, metabolite, salt, crystalline form, non-crystalline form, hydrate or solvate thereof.

2. position according to claim 1, comprising one or more compounds of Formula I selected from alpha-tocotrienol quinone, beta-tocotrienol quinone, gamma-tocotrienol quinone, and delta-tocotrienol quinone, or any stereoisomer, or mixture of stereoisomers thereof.

3. The composition according to claim 1, comprising alpha-tocotrienol quinone or any stereoisomer, or mixture of stereoisomers thereof.

4. A composition comprising one or more compounds of Formula I according to claim 1, in an amount effective for preventing, reducing, ameliorating or treating diabetes, or to stabilize the level of blood sugar, stop the increase in level of blood sugar, reverse the increase in the level of blood sugar, or reduce the level of blood sugar in a subject with abnormal glucose tolerance and metabolism in need of such treatment.

5. position according to claim 4, comprising one or more compounds of Formula I selected from alpha-tocotrienol quinone, beta-tocotrienol quinone, gamma-tocotrienol quinone, and delta-tocotrienol quinone or any stereoisomer, or mixture of stereoisomers thereof.

6. The composition according to claim 5, comprising alpha-tocotrienol quinone, or any stereoisomer, or mixture of stereoisomers thereof.

7. A composition comprising one or more compounds of Formula I according to claim 1, in an amount effective for preventing or treating a condition requiring increasing insulin sensitivity or reducing insulin resistance in a subject with abnormal glucose tolerance and metabolism in need of such treatment.

8. The composition according to claim 7, comprising one or more compounds of Formula I selected from alpha-tocotrienol quinone, beta-tocotrienol quinone, gamma-tocotrienol quinone, and delta-tocotrienol quinone or any stereoisomer, or mixture of stereoisomers thereof.

9. The composition according to claim 8, comprising alpha-tocotrienol quinone, or any stereoisomer, or mixture of stereoisomers thereof.

10. A composition comprising one or more compounds of Formula I according to claim 1, in an amount effective for preventing, reducing, ameliorating or treating vascular disease complications, including retinopathy and nephropathy, in a subject with abnormal glucose tolerance and metabolism in need of such treatment.

11. The composition according to claim 10, comprising one or more compounds of Formula I selected from alpha-tocotrienol quinone, beta-tocotrienol quinone, gamma-tocotrienol quinone, and delta-tocotrienol quinone or any stereoisomer, or mixture of stereoisomers thereof.

12. The composition according to claim 11, comprising alpha-tocotrienol quinone, or any stereoisomer, or mixture of stereoisomers thereof.

13. A composition comprising one or more compounds of Formula I according to claim 1, in an amount effective for preventing, reducing, ameliorating or treating inflammation in a subject with abnormal glucose tolerance and metabolism in need of such treatment.

14. The composition according to claim 13, comprising one or more compounds of Formula I selected from alpha-tocotrienol quinone, beta-tocotrienol quinone, gamma-tocotrienol quinone, and delta-tocotrienol quinone or any stereoisomer, or mixture of stereoisomers thereof.

15. The composition according to claim 14, comprising alpha-tocotrienol quinone, or any stereoisomer, or mixture of stereoisomers thereof.

16. A formulation comprising one or more compounds of Formula I according to claim 1, and, optionally, a pharmaceutically acceptable carrier, diluent or excipient, in an amount effective for improving blood glucose control in a subject with abnormal glucose tolerance and metabolism in need of such treatment.

17. A formulation comprising one or more compounds of Formula I according to claim 1, and, optionally, a pharmaceutically acceptable carrier, diluent or excipient, in an amount effective for preventing, reducing, ameliorating or treating diabetes, or to stabilize the level of blood sugar, stop the increase in level of blood sugar, reverse the increase in the level of blood sugar, or reduce the level of blood sugar in a subject with abnormal glucose tolerance and metabolism in need of such treatment.

18. A formulation comprising one or more compounds of Formula I according to claim 1, and, optionally, a pharmaceutically acceptable carrier, diluent or excipient, in an amount effective for preventing or treating a condition requiring increasing insulin sensitivity or reducing insulin resistance in a subject with abnormal glucose tolerance and metabolism in need of such treatment.

19. A formulation comprising one or more compounds of Formula I according to claim 1, and, optionally, a pharmaceutically acceptable carrier, diluent or excipient, in an amount effective for preventing, reducing, ameliorating or treating vascular disease complications, including retinopathy and nephropathy, in a subject with abnormal glucose tolerance and metabolism in need of such treatment.

20. A formulation comprising one or more compounds of Formula I according to claim 1, and, optionally, a pharmaceutically acceptable carrier, diluent or excipient, in an amount effective for preventing or treating inflammation in a subject with abnormal glucose tolerance and metabolism in need of such treatment.

21. A method for improving blood glucose control in a subject with abnormal glucose tolerance and metabolism in need of such treatment which comprises administering an effective amount of one or more compounds of Formula I of claim 1 and, optionally, a pharmaceutically acceptable carrier, diluent or excipient.

22. The method according to claim 21, comprising administering an effective amount of one or more compounds of Formula I selected from alpha-tocotrienol quinone, beta-tocotrienol quinone, gamma-tocotrienol quinone, and delta-tocotrienol quinone, or any stereoisomer, or mixture of stereoisomers thereof.

23. The method according to claim 22, comprising administering an effective amount of alpha-tocotrienol quinone, or any stereoisomer, or mixture of stereoisomers thereof.

24. A method for preventing, reducing, ameliorating or treating diabetes, or to stabilize the level of blood sugar, stop the increase in level of blood sugar, reverse the increase in the level of blood sugar, or reduce the level of blood sugar in a subject with abnormal glucose tolerance and metabolism in need of such treatment, which comprises administering an effective amount of one or more compounds of Formula I of claim 1 and, optionally, a pharmaceutically acceptable carrier, diluent or excipient.

25. The method according to claim 24, comprising administering an effective amount of one or more compounds of Formula I selected from alpha-tocotrienol quinone, beta-tocotrienol quinone, gamma-tocotrienol quinone, and delta-tocotrienol quinone, or any stereoisomer, or mixture of stereoisomers thereof.

26. The method according to claim 25, comprising administering an effective amount of alpha-tocotrienol quinone, or any stereoisomer, or mixture of stereoisomers thereof.

27. A method for preventing or treating a condition requiring increasing insulin sensitivity or reducing insulin resistance in a subject with abnormal glucose tolerance and metabolism in need of such treatment which comprises administering an effective amount of one or more compounds of Formula I and, optionally, a pharmaceutically acceptable carrier, diluent or excipient according to claim 1.

28. The method according to claim 27, comprising administering an effective amount of one or more compounds of Formula I selected from alpha-tocotrienol quinone, beta-tocotrienol quinone, gamma-tocotrienol quinone, and delta-tocotrienol quinone, or any stereoisomer, or mixture of stereoisomers thereof.

29. The method according to claim 28, comprising administering an effective amount of alpha-tocotrienol quinone, or any stereoisomer, or mixture of stereoisomers thereof.

30. A method for preventing, reducing, ameliorating or treating vascular disease complications of diabetes, including retinopathy and nephropathy, in a subject with abnormal glucose tolerance and metabolism in need of such treatment, which comprises administering an effective amount of one or more compounds of Formula I of claim 1, and, optionally, a pharmaceutically acceptable carrier, diluent or excipient.

31. The method according to claim 30, comprising administering an effective amount of one or more compounds of Formula I selected from alpha-tocotrienol quinone, beta-tocotrienol quinone, gamma-tocotrienol quinone, and delta-tocotrienol quinone, or any stereoisomer, or mixture of stereoisomers thereof.

32. The method according to claim 31, comprising administering an effective amount of alpha-tocotrienol quinone, or any stereoisomer, or mixture of stereoisomers thereof.

33. A method for preventing, reducing, ameliorating or treating inflammation in a subject with abnormal glucose tolerance and metabolism in need of such treatment, which comprises administering an effective amount of one or more compounds of Formula I of claim 1, and, optionally, a pharmaceutically acceptable carrier, diluent or excipient.

34. The method according to claim 33, comprising administering an effective amount of one or more compounds of Formula I selected from alpha-tocotrienol quinone, beta-tocotrienol quinone, gamma-tocotrienol quinone, and delta-tocotrienol quinone, or any stereoisomer, or mixture of stereoisomers thereof.

35. The method according to claim 34, comprising administering an effective amount of alpha-tocotrienol quinone, or any stereoisomer, or mixture of stereoisomers thereof.

36. The method of claim 21, wherein the formulation is administered orally or enterally.

37. The method of claim 24, wherein the formulation is administered orally or enterally.

38. The method of claim 27, wherein the formulation is administered orally or enterally.

39. The method of claim 30, wherein the formulation is administered orally or enterally.

40. The method of claim 33, wherein the formulation is administered orally or enterally.
